(19) United States
(12) Patent Application Publication
(10) Pub. No.: US 2012/0040966 A1
(43) Pub. Date: Feb. 16, 2012

(54) ANTI-VIRAL COMPOUNDS, TREATMENT, AND ASSAY

(75) Inventors: Joseph A. Maddry, Birmingham, AL (US); Bill Severson, Birmingham, AL (US); Colleen B. Jonsson, Louisville, KY (US); James W. Noah, Sterrett, AL (US); Diana L. Noah, Sterrett, AL (US); Xi Chen, Hoover, AL (US)

(73) Assignee: Southern Research Institute Office of Commercialization and Intellectual Prop, Birmingham, AL (US)

(21) Appl. No.: 13/142,678

(22) PCT Filed: Dec. 28, 2009
(86) PCT No.: PCT/US2009/069556

§ 371 Date: Oct. 31, 2011

Related U.S. Application Data
(60) Provisional application No. 61/141,161, filed on Dec. 29, 2008.

(51) Int. Cl. A61K 31/55; C07D 405/10; C07D 401/04; C07D 491/052; C07D 495/04; A61P 31/16; A61K 31/517; A61K 31/519; A61K 31/5377; C12Q 1/70; A61P 31/12; A61P 31/14; C07D 239/70; C07D 471/04

(52) U.S. Cl. 514/215; 544/249; 544/250; 544/115; 540/586; 514/267; 514/232.8; 435/5

(57) ABSTRACT

The compounds provided herein are suitable for inhibiting a virus in a patient and for treating a patient suffering from a viral infection such as an influenza virus. Also provided is a method for screening of compounds for use in treating and preventing viral infections. The assay screens compounds for activity against an influenza virus by measuring the cytopathogenic effect (CPE) of the compound on influenza infected-cells using percent cell viability as the end point.

### TECHNICAL FIELD

The present disclosure relates to a method for treating human and/or animals infected with a virus including various respiratory viruses, including members of families Orthomyxoviridae, (Influenza A and B viruses (all HN serotypes)), Paramyxoviridae, (Respiratory syncytial virus (RSV), human metapneumovirus (HMPV), human parainfluenza virus (HPIV), measles virus, and mumps virus), Bunyaviridae, (hantavirus, sin nombre virus (SNV), Rift Valley Fever virus (RVFV)), Coronaviridae, SARS-CoV, Adenoviridae, adenovirus-associated respiratory viruses, and Picornaviridae, (coxsackie A viruses (CA), coxsackie B viruses (CB), echoviruses and rhinoviruses)). The applications of this disclosure also include those situations in which preventing virus-induced cytopathic effect (CPE) can result in the protection against infections. The present disclosure also relates to those compounds of this disclosure that are novel. The present disclosure also relates to a CPE-based assay that will assess virus—induced CPE for screening of compounds for treating viral diseases or inhibiting a virus.

### BACKGROUND OF DISCLOSURE

Influenza virus (Family Orthomyxoviridae) is an important human pathogen that causes substantial morbidity and mortality with approximately 36,000 flu-related deaths in the US each year [1]. Influenza A viruses, which also infect a wide number of avian and mammalian species including pigs, horses, and humans pose a considerable threat in terms of epidemic and pandemic potential. In the twentieth century three influenza pandemics have occurred. Approximately 20 to 40 percent of the world's population became ill during the catastrophic 1918 flu pandemic which killed 675,000 people in the U.S. and an estimated 20-50 million people worldwide. The “Asian” flu pandemic of 1957 resulted in the deaths of approximately 69,800 people in the U.S. and 2.0 to 7.4 million worldwide [2]. Approximately 38,000 people living in the USA died during the third and mildest pandemic in the 20th century, the “Hong Kong” flu pandemic of 1968. The pandemics of 1957 and 1968 were quickly identified due to increased surveillance for flu outbreaks and technological advances in influenza biology. The survival rate for the 1968 pandemic was greatly increased due to improved medical care and the development of antimicrobials to protect against secondary bacterial infections. In 1997, the Special Administrative Region of China in Hong Kong garnered worldwide attention when an epidemic of highly pathogenic avian influenza (H5N1) virus was transmitted from poultry to humans resulting in 18 human cases, of which six were fatal [3]. Avian influenza (H5N1) may adapt into a strain that is highly communicable to humans. Hence the global public health and research communities have paid special attention to the epidemiology, ecology and microbiology of these newly emerging influenza H5N1 strains. On Jun. 11, 2009, the Health Organization raised the worldwide pandemic alert level to Phase 6 in response to the human “swine flu” outbreak of a novel H1N1 strain. The current health burden of epidemic influenza, and the “swine flu” H1N1 pandemic, has increased effort towards the discovery and development of antivirals and vaccines for the treatment of influenza disease.

### SUMMARY OF DISCLOSURE

The present disclosure relates to compounds represented by the following formulas:

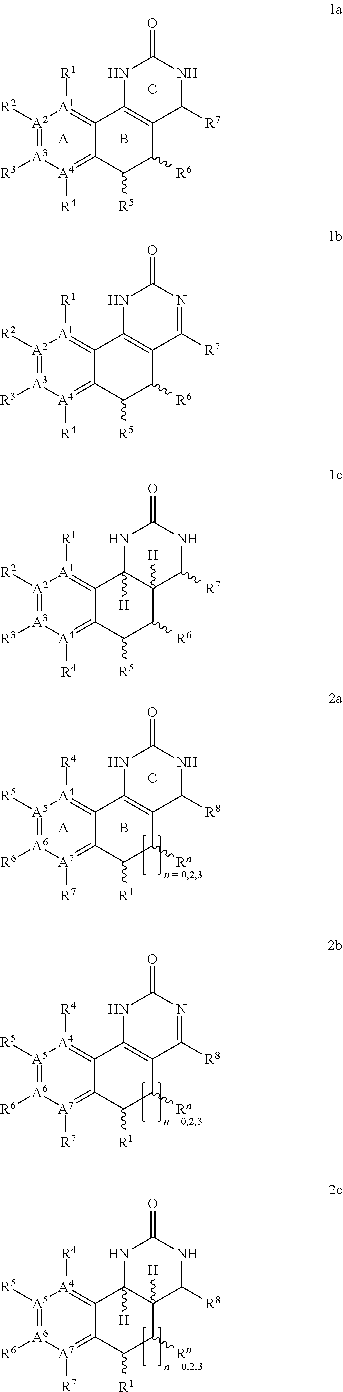

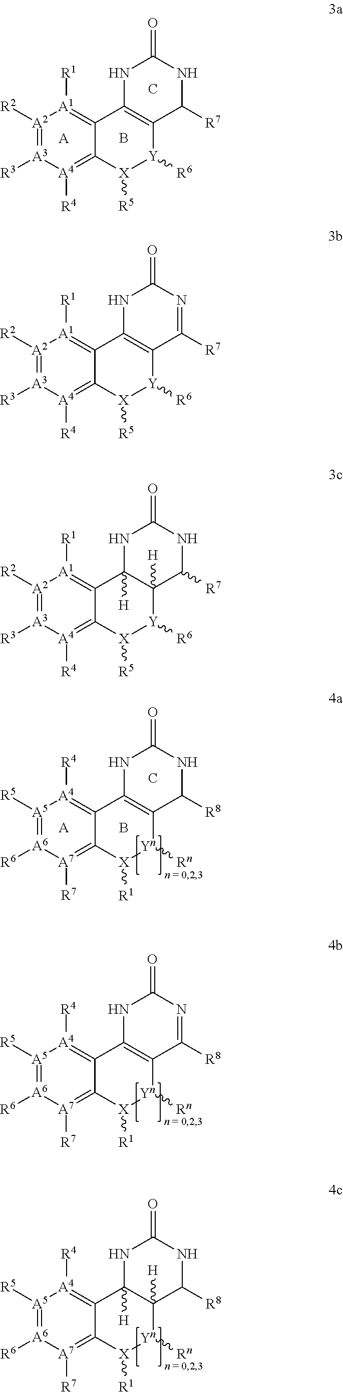

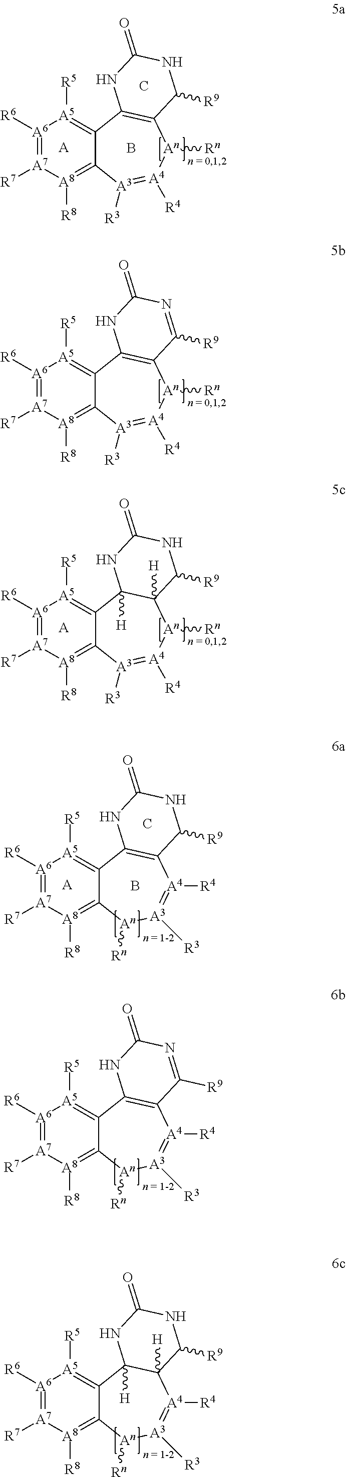

or their stereoisomerically pure forms, or a pharmaceutically acceptable salt thereof, a solvate thereof, a prodrug thereof, and mixtures thereof.

In all cases, the ring designated C may be represented in multiple equivalent tautomeric forms, as exemplified by Structures 7.

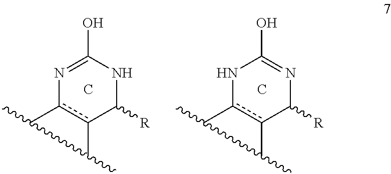

In formulas 1a, 1b, and 1c, atoms labeled A¹ thru A⁴ are independently chosen from C or N. Whenever any of the Aⁱ=C (i=1-4), then those atoms may optionally and independently be substituted by the corresponding substituents Rⁱ (i=1-4), where each of the Rⁱ are individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido.

Similarly, the substituents R⁵, R⁶, and R⁷ are individually and independently selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido.

In formulas 2a, 2b, and 2c, atoms labeled A⁴ thru A⁷ are independently chosen from C or N. Whenever any of the Aⁱ=C (i=4-7), then those atoms may optionally and independently be substituted by the corresponding substituents Rⁱ (i=4-7), where each of the Rⁱ are individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido.

Similarly, the substituents R¹, and, when present, R² and R³, are individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido.

In formulas 3a, 3b, and 3c, atoms labeled A¹ thru A⁴ are independently chosen from C or N. Whenever any of the Aⁱ=C (i=1-4), then those atoms may optionally and independently be substituted by the corresponding substituents Rⁱ (i=1-4), where each of the Rⁱ are individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido.

The atoms labeled X and Y are independently chosen from the group C, N, O, or S, where at least one of X or Y is not C. When X═C or N, or Y═C, N, then they may be optionally and independently substituted by substituents R⁵ and R⁶, respectively. When either R⁵ or R⁶ are bound to C, they may be individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido. When either R⁵ or R⁶ are bound to N, they may be individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, amino, mono- or disubstituted amino (hydrazino), alkylthio, carbonyl and alkyl- or aryl-substituted carbonyl (amide), carboxyl and alkoxycarbonyl (carbamate), and aminocarbonyl (urea). R⁷ may be individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido.

In formulas 4a, 4b, and 4c, atoms labeled A⁴ thru A⁷ are independently chosen from C or N. Whenever any of the Aⁱ=C (i=4-7), then those atoms may optionally and independently be substituted by the corresponding substituents Rⁱ (i=4-7), where each of the Rⁱ are individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido.

When n=2 or 3, the atoms labeled X and any of those labeled Yⁿ are independently chosen from the group C, N, O, or S. In this case, each of X and any of the Yⁿ may be optionally and independently substituted by substituents R¹ and Rⁿ, respectively. When either R¹ or any of the Rⁿ are bound to C, they may be individually and independently selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido. When either R¹ or any of the Rⁿ are bound to N, they may be individually and independently selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, amino, mono- or disubstituted amino (hydrazino), alkylthio, carbonyl and alkyl- or aryl-substituted carbonyl (amide), carboxyl and alkoxycarbonyl (carbamate), and aminocarbonyl (urea). R⁸ may be individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido.

In formulas 5a, 5b, and 5c, atoms labeled A³ thru A⁸ are independently chosen from C or N. Whenever any of the Aⁱ=C (i=3-8), then those atoms may optionally and independently be substituted by the corresponding substituents Rⁱ (i=3-8), where each of the Rⁱ are individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido.

When n=1 or 2, any of the atom(s) labeled Aⁿ are independently chosen from the group C, N, O, or S. In this case, each of the atoms labeled Aⁿ may be optionally and independently substituted by substituents Rⁿ, respectively. When any of the Rⁿ are bound to C, they may be individually and independently selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido. When any of the Rⁿ are bound to N, they may be individually and independently selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, amino, mono- or disubstituted amino (hydrazino), alkylthio, carbonyl and alkyl- or aryl-substituted carbonyl (amide), carboxyl and alkoxycarbonyl (carbamate), and aminocarbonyl (urea). R⁹ may be individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido.

In formulas 6a, 6b, and 6c, atoms labeled A³ thru A⁸ are independently chosen from C or N. Whenever any of the Aⁱ=C (i=3-8), then those atoms may optionally and independently be substituted by the corresponding substituents Rⁱ (i=3-8), where each of the Rⁱ are individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido.

When n=1 or 2, any of the atom(s) labeled Aⁿ are independently chosen from the group C, N, O, or S. In this case, any of the atoms labeled Aⁿ may be optionally and independently substituted by substituents Rⁿ, respectively. When any of the Rⁿ are bound to C, they may be individually and independently selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido.

When any of the Rⁿ are bound to N, they may be individually and independently selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, amino, mono- or disubstituted amino (hydrazino), alkylthio, carbonyl and alkyl- or aryl-substituted carbonyl (amide), carboxyl and alkoxycarbonyl (carbamate), and aminocarbonyl (urea). R⁹ may be individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido.

The present disclosure also relates to a method for inhibiting a virus in a host or patient by administering to the host or patient in an amount effective for treating the host or patient at least one of the disclosed compounds.

The hosts or patients to be treated according to this disclosure include humans and animals such as zoo or exotic animals, food animals (e.g. cattle, sheep and goats) and companion animals (e.g. dogs and cats).

The present disclosure also relates to a cytopathogenic-based assay that will assess influenza virus-induced CPE in Madin Darby canine kidney (MDCK) cells to determine adenosine triphosphate (ATP) concentration using a luciferase reporter for screening for compounds that can be used in treating infectious diseases.

Still other objects and advantages of the present disclosure will become readily apparent by those skilled in the art from the following detailed description, wherein it is shown and described preferred embodiments, simply by way of illustration of the best mode contemplated. As will be realized the disclosure is capable of other and different embodiments, and its several details are capable of modifications in various obvious respects, without departing from the disclosure. Accordingly, the description is to be regarded as illustrative in nature and not as restrictive.

### BRIEF DESCRIPTION OF THE FIGURES

FIG. 1: FIG. 1 presents pK data for eight compounds having in vitro activity against influenza A viruses.

### BEST AND VARIOUS MODES FOR CARRYING OUT DISCLOSURE

The present disclosure relates compounds represented by the formulas:

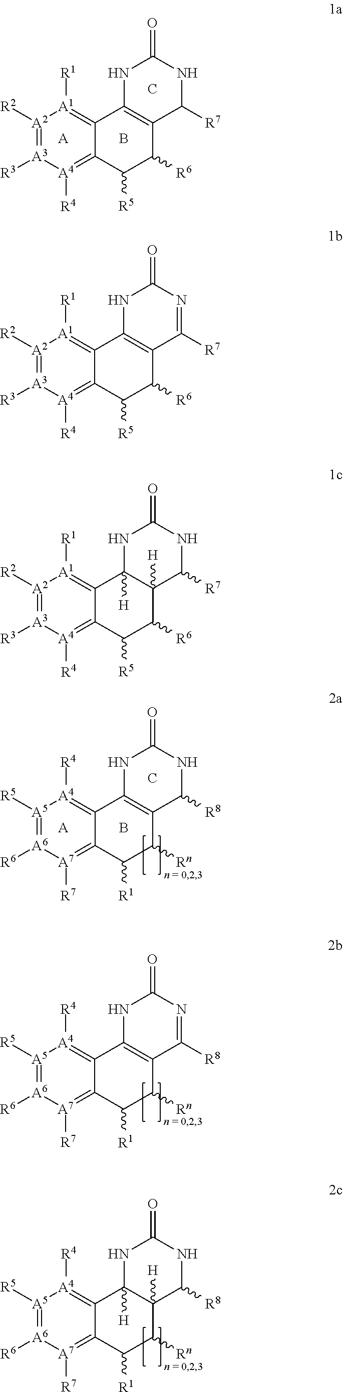

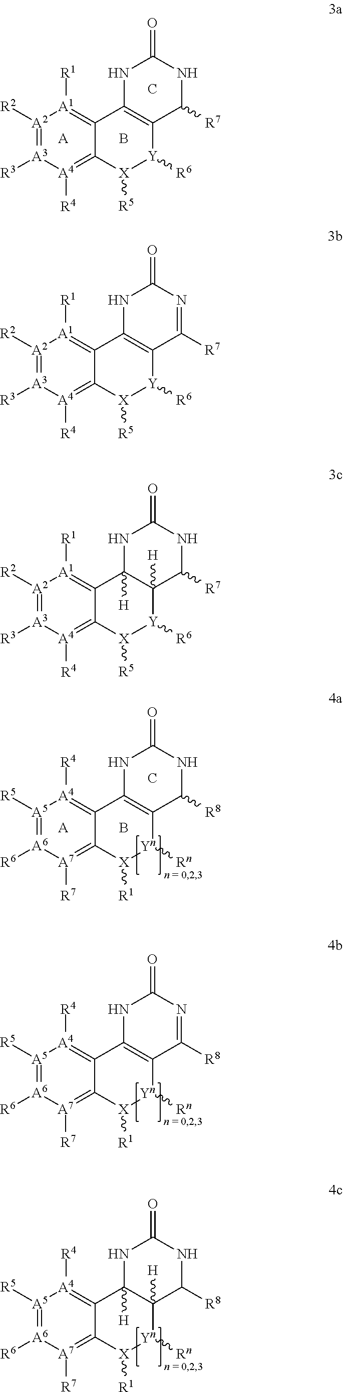

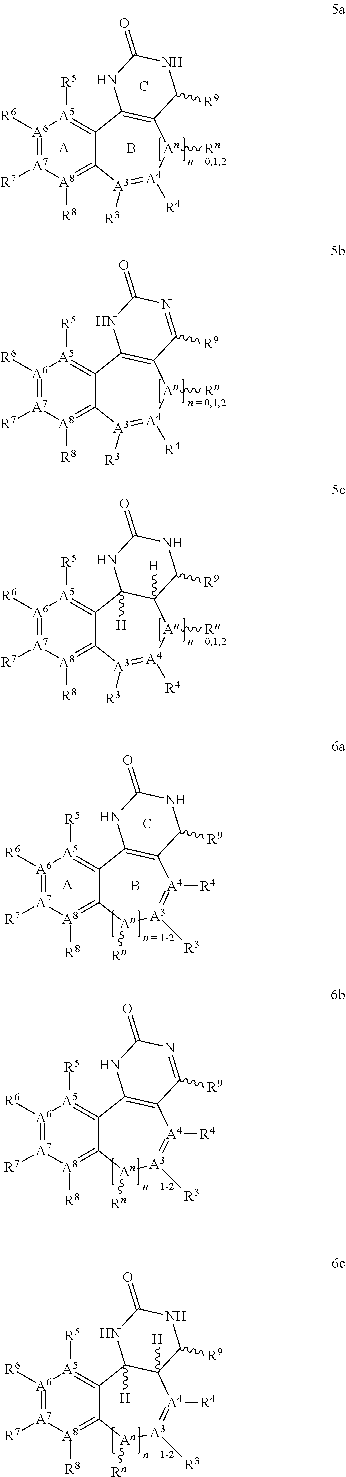

or their stereoisomerically pure forms, or a pharmaceutically acceptable salt thereof, a solvate thereof, a prodrug thereof, and mixtures thereof. In all cases, the ring designated C may be represented in multiple equivalent tautomeric forms, as exemplified by Structures 7.

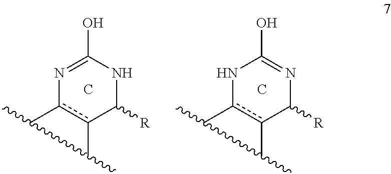

In formulas 1a, 1b, and 1c, atoms labeled A¹ thru A⁴ are independently chosen from C or N. Whenever any of the Aⁱ=C (i=1-4), then those atoms may optionally and independently be substituted by the corresponding substituents Rⁱ (i=1-4), where each of the Rⁱ are individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy halogen, azido, cyano, nitro, alkylthio, carboxyl and corresponding esters, carboxamido and amino and mono- or disubstituted amino including amido.

Similarly, the substituents R⁵, R⁶, and R⁷ are individually and independently selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido.

In formulas 2a, 2b, and 2c, atoms labeled A⁴ thru A⁷ are independently chosen from C or N. Whenever any of the Aⁱ=C (i=4-7), then those atoms may optionally and independently be substituted by the corresponding substituents Rⁱ (i=4-7), where each of the Rⁱ are individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido. Similarly, the substituents R¹, and, when present, R² and R³, are individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido.

In formulas 3a, 3b, and 3c, atoms labeled A¹ thru A⁴ are independently chosen from C or N. Whenever any of the Aⁱ=C (i=1-4), then those atoms may optionally and independently be substituted by the corresponding substituents Rⁱ (i=1-4), where each of the Rⁱ are individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido.

The atoms labeled X and Y are independently chosen from the group C, N, O, or S, where at least one of X or Y is not C. When X═C or N, or Y═C, N, then they may be optionally and independently substituted by substituents R⁵ and R⁶, respectively. When either R⁵ or R⁶ are bound to C, they may be individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy halogen, azido, cyano, nitro, alkylthio, carboxyl and corresponding esters, carboxamido and amino and mono- or disubstituted amino including amido. When either R⁵ or R⁶ are bound to N, they may be individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, amino, mono- or disubstituted amino (hydrazino), alkylthio, carbonyl and alkyl- or aryl-substituted carbonyl (amide), carboxyl and alkoxycarbonyl (carbamate), and aminocarbonyl (urea). R⁷ may be individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido.

In formulas 4a, 4b, and 4c, atoms labeled A⁴ thru A⁷ are independently chosen from C or N. Whenever any of the Aⁱ=C (i=4-7), then those atoms may optionally and independently be substituted by the corresponding substituents Rⁱ (i=4-7), where each of the Rⁱ are individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido.

When n=2 or 3, the atoms labeled X and any of those labeled Yⁿ are independently chosen from the group C, N, O, or S. In this case, each of X and any of the Yⁿ may be optionally and independently substituted by substituents R¹ and Rⁿ, respectively. When either R¹ or any of the Rⁿ are bound to C, they may be individually and independently selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido. When either R¹ or any of the Rⁿ are bound to N, they may be individually and independently selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, amino, mono- or disubstituted amino (hydrazino), alkylthio, carbonyl and alkyl- or aryl-substituted carbonyl (amide), carboxyl and alkoxycarbonyl (carbamate), and aminocarbonyl (urea). R⁸ may be individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido.

In formulas 5a, 5b, and 5c, atoms labeled A³ thru A⁸ are independently chosen from C or N. Whenever any of the Aⁱ=C (i=3-8), then those atoms may optionally and independently be substituted by the corresponding substituents Rⁱ (i=3-8), where each of the Rⁱ are individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido.

When n=1 or 2, any of the atom(s) labeled Aⁿ are independently chosen from the group C, N, O, or S. In this case, each of the atoms labeled Aⁿ may be optionally and independently substituted by substituents Rⁿ, respectively. When any of the Rⁿ are bound to C, they may be individually and independently selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido. When any of the Rⁿ are bound to N, they may be individually and independently selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, amino, mono- or disubstituted amino (hydrazino), alkylthio, carbonyl and alkyl- or aryl-substituted carbonyl (amide), carboxyl and alkoxycarbonyl (carbamate), and aminocarbonyl (urea). R⁹ may be individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido.

In formulas 6a, 6b, and 6c, atoms labeled A³ thru A⁸ are independently chosen from C or N. Whenever any of the Aⁱ=C (i=3-8), then those atoms may optionally and independently be substituted by the corresponding substituents Rⁱ (i=3-8), where each of the Rⁱ are individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido.

When n=1 or 2, any of the atom(s) labeled Aⁿ are independently chosen from the group C, N, O, or S. In this case, any of the atoms labeled Aⁿ may be optionally and independently substituted by substituents Rⁿ, respectively. When any of the Rⁿ are bound to C, they may be individually and independently selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido.

When any of the Rⁿ are bound to N, they may be individually and independently selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, amino, mono- or disubstituted amino (hydrazino), alkylthio, carbonyl and alkyl- or aryl-substituted carbonyl (amide), carboxyl and alkoxycarbonyl (carbamate), and aminocarbonyl (urea). R⁹ may be individually selected from the group consisting of hydrogen, substituted or unsubstituted alkyl including trifluoromethyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino including amido.

Listed below are definitions of various terms used to describe this invention. These definitions apply to the terms as they are used throughout this specification, unless otherwise limited in specific instances, either individually or as part of a larger group. Also, in the formulas described and claimed herein, it is intended that when any symbol appears more than once in a particular formula or substituent, its meaning in each instance is independent of the other.

The term “comprising” (and its grammatical variations) as used herein is used in the inclusive sense of “having” or “including” and not in the exclusive sense of “consisting only of.” The term “consisting essentially of” as used herein is intended to refer to including that which is explicitly recited along with what does not materially affect the basic and novel characteristics of that recited or specified.

The terms “a” and “the” as used herein are understood to encompass the plural as well as the singular.

The terms “effective amount” or “therapeutically effective amount” refer to an amount of the compound of the invention sufficient to provide a benefit in the treatment or prevention of viral disease, to delay or minimize symptoms associated with viral infection or viral-induced disease, or to cure or ameliorate the disease or infection or cause thereof. In particular, a therapeutically effective amount means an amount sufficient to provide a therapeutic benefit in vivo. Used in connection with an amount of a compound of the invention, the term preferably encompasses a non-toxic amount that improves overall therapy, reduces or avoids symptoms or causes of disease, or enhances the therapeutic efficacy of or synergies with another therapeutic agent

The term “treating” refers to relieving the disease, disorder, or condition, i.e., causing regression of the disease, disorder, and/or condition; preventing a disease, disorder, or condition from occurring in an animal that may be predisposed to the disease, disorder and/or condition, but has not yet been diagnosed as having it; and/or inhibiting the disease, disorder, or condition, i.e., arresting its development.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

“Pharmaceutically acceptable salts” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids.

Typical inorganic acids used to form such salts include hydrochloric, hydrobromic, hydroiodic, nitric, sulfuric, phosphoric, hypophosphoric and the like. Salts derived from organic acids, such as aliphatic mono and dicarboxylic acids, phenyl substituted alkonic acids, hydroxyalkanoic and hydroxyalkandioic acids, aromatic acids, aliphatic and aromatic sulfonic acids, may also be used. Such pharmaceutically acceptable salts thus include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, β-hydroxybutyrate, butyne-1,4-dioate, hexyne-1,4-dioate, cabrate, caprylate, chloride, cinnamate, citrate, formate, fumarate, glycollate, heptanoate, hippurate, lactate, malate, maleate, hydroxymaleate, malonate, mandelate, mesylate, nicotinate, isonicotinate, nitrate, oxalate, phthalate, teraphthalate, phosphate, monohydrogenphosphate, dihydrogenphosphate, metaphosphate, pyrophosphate, propiolate, propionate, phenylpropionate, salicylate, sebacate, succinate, suberate, sulfate, bisulfate, pyrosulfate, sulfite, bisulfite, sulfonate, benzene-sulfonate, p-bromobenzenesulfonate, chlorobenzenesulfonate, ethanesulfonate, 2-hydroxyethanesulfonate, methanesulfonate, naphthalene-1-sulfonate, naphthalene-2-sulfonate, p-toluenesulfonate, xylenesulfonate, tartarate, and the like.

Bases commonly used for formation of salts include ammonium hydroxide and alkali and alkaline earth metal hydroxides, carbonates, as well as aliphatic and primary, secondary and tertiary amines, aliphatic diamines. Bases especially useful in the preparation of addition salts include sodium hydroxide, potassium hydroxide, ammonium hydroxide, potassium carbonate, methylamine, diethylamine, and ethylene diamine.

A “prodrug” is a compound that is converted within the body into its active form that has a medical effect. Prodrugs may be useful when the active drug may be too toxic to administer systemically, the active drug is absorbed poorly by the digestive tract, or the body breaks down the active drug before it reaches its target. Methods of making prodrugs are disclosed in Hans Bundgaard, DESIGN OF PRODRUGS (Elsevier Science Publishers B.V. 1985), which is incorporated herein by reference in its entirety.

Prodrug forms of the compounds bearing various nitrogen functions (amino, hydroxyamino, hydrazino, guanidino, amidino, amide, etc.) may include the following types of derivatives where each R group individually may be hydrogen, substituted or unsubstituted alkyl, aryl, alkenyl, alkynyl, heterocycle, alkylaryl, aralkyl, aralkenyl, aralkenyl, cycloalkyl or cycloalkenyl groups as defined below:

carboxamides (—NHC(O)R);

carbamates (—NHC(O)OR);

(acyloxy)alkyl carbamates (—NHC(O)OROC(O)R);

enamines (—NHCR(═CHCRO₂R) or —NHCR(═CHCRONR₂));

Schiff bases (—N═CR₂); and

Mannich bases from carboxamide compounds (RCONHCH₂NR₂).

Preparations of such prodrug derivatives are discussed in various literature sources (examples are: Alexander et al., J. Med. Chem. 1988, 31, 318; Aligas-Martin et al., PCT WO pp/41531, p. 30). The nitrogen function converted in preparing these derivatives is one (or more) of the nitrogen atoms of a compound of the invention.

Prodrug forms of carboxyl-bearing compounds of the disclosure include esters (—CO₂R) where the R group corresponds to any alcohol whose release in the body through enzymatic or hydrolytic processes would be at pharmaceutically acceptable levels.

Another prodrug derived from a carboxylic acid form of the disclosure may be a quaternary salt type:

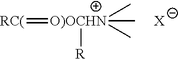

of structure described by Bodor et al., J. Med. Chem. 1980, 23, 469.

It is of course understood that the compounds of the present disclosure relate to all optical isomers and stereo-isomers at the various possible atoms of the molecule.

“Solvates” refers to the compound formed by the interaction of a solvent and a solute and includes hydrates. Solvates are usually crystalline solid adducts containing solvent molecules within the crystal structure, in either stoichiometric or nonstoichiometric proportions.

The term “halogen” or “halo” refers to fluorine, chlorine, bromine and iodine.

The term “aryl” refers to monocyclic or bicyclic aromatic hydrocarbon groups having 6 to 12 carbon atoms in the ring portion, such as phenyl, naphthyl, biphenyl and diphenyl groups, each of which may be substituted. The aromatic or aryl groups are more typically phenyl and alkyl substituted aromatic groups (aralkyl) such as phenyl C₁₋₃ alkyl and benzyl.

The term “aralkyl” or “arylalkyl” refers to an aryl group bonded directly through an alkyl group, such as benzyl or phenethyl.

The term “substituted aryl” or “substituted alkylaryl” refers to an aryl group or alkylaryl group substituted by, for example, one to four substituents such as aryl, substituted aryl, heterocycle, substituted heterocycle, alkyl; substituted alkyl, halo, trifluoromethoxy, trifluoromethyl, hydroxy, alkoxy, azido, cycloalkyloxy, heterocyclooxy, alkanoyl, alkanoyloxy, amino, alkylamino, aralkylamino, hydroxyalkyl, aminoalkyl, azidoalkyl, alkenyl, alkynyl, allenyl, cycloalkylamino, heterocycloamino, dialkylamino, alkanoylamino, thiol, alkylthio, cycloalkylthio, heterocyclothio, ureido, nitro, cyano, carboxy, carboxyalkyl, carbamyl, alkoxycarbonyl, alkylthiono, arylthiono, alkysulfonyl, sulfonamido, aryloxy and the like. The substituent may be further substituted by halo, hydroxy, alkyl, alkoxy, aryl, substituted aryl, substituted alkyl or aralkyl. “Substituted benzyl” refers to a benzyl group substituted by, for example, any of the groups listed above for substituted aryl.

The term “cycloalkyl” refers to optionally substituted, saturated cyclic hydrocarbon ring systems, preferably containing 1 to 3 rings and 3 to 7 carbons per ring which may be further fused with an unsaturated C₃-C₇ carbocyclic ring. Exemplary groups include cyclopropyl, cyclobutyl, cyclopentyl, cyclohexyl, cycloheptyl, cyclooctyl, cyclodecyl, cyclododecyl and adamantyl. Exemplary substituents include one or more alkyl groups as described above, or one or more groups described above as alkyl substituents.

The term “alkyl” refers to straight or branched chain unsubstituted hydrocarbon groups of 1 to 20 carbon atoms, and more typically 1 to 8 carbon atoms and even more typically unsubstituted alkyl groups of 1 to 4 carbon atoms. Examples of suitable alkyl groups include methyl, ethyl and propyl. Examples of branched alkyl groups include isopropyl and t-butyl.

The terms “heterocycle”, “heterocyclic” and “heterocyclo” refer to an optionally substituted, fully saturated or unsaturated, aromatic or nonaromatic cyclic group, for example, which is a 4 to 7 membered monocyclic, 7 to 11 membered bicyclic, or 10 to 15 membered tricyclic ring system, which has at least one heteroatom and at least one carbon atom in the ring. Each ring of the heterocyclic group containing a heteroatom may have 1, 2 or 3 heteroatoms selected from nitrogen atoms, oxygen atoms and sulfur atoms, where the nitrogen and sulfur heteroatoms may also optionally be oxidized and the nitrogen heteroatoms may also optionally be quaternized. The heterocyclic group may be attached at any heteroatom or carbon atom. Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), piperidinyl, pyrrolidine, tetrahydrofuranyl, furyl, furanyl, pyridyl, pyrimidyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzimidazolyl, purinyl, carbazolyl, oxazolyl, thiazolyl, isothiazolyl, 1,2,4-thiadiazolyl, isooxazolyl, pyrrolyl, quinazolinyl, cinnolinyl, phthalazinyl, xanthinyl, hypoxanthinyl, thiophene, furan, isopyrrole, 1,2,3-triazole, 1,2,4-triazole, oxazole, thiazole, pyrimidine, aziridines, thiazole, 1,2,3-oxadiazole, thiazine, pyrrolidine, oxaziranes, morpholinyl, pyrazolyl, pyridazinyl, pyrazinyl, quinoxalinyl, xanthinyl, hypoxanthinyl, pteridinyl, 5-azacytidinyl, 5-azauracilyl, triazolopyridinyl, imidazolopyridinyl, pyrrolopyrimidinyl, pyrazolopyrimidinyl, adenine, N6-alkylpurines, N6-benzylpurine, N6-halopurine, N6-vinylpurine, N6-acetylenic purine, N6-acyl purine, N6-hydroxyalkyl purine, N6-thioalkyl purine, thymine, cytosine, 6-azopyrimidine, 2-mercaptopyrimidine, uracil, N5-alkyl-pyrimidines, N5-benzylpyrimidines, N5-halopyrimidines, N5-vinyl-pyrimidine, N5-acetylenic pyrimidine, N5-acyl pyrimidine, N5-hydroxyalkyl purine, and N6-thioalkyl purine, and isoxazolyl. The heteroaromatic and heterocyclic moieties can be optionally substituted as described above for aryl, including substituted with one or more substituents selected from hydroxyl, amino, alkylamino, arylamino, alkoxy, aryloxy, alkyl, heterocycle, halo, carboxy, acyl, acyloxy, amido, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991.

The term “amino” as used herein refers to the group —NH₂.

When any of the above groups are substituted, unless stated otherwise, they are typically substituted with at least one member selected from the group consisting of alkyl, hydroxyl, amino, halo and halogenated alkyl and more typically a fluoroalkyl such as trifluoromethyl.

### Representative Compounds

Representative compounds according to the present disclosure along with test data are disclosed in the following Table I:

TABLE I

| | | | | H5N1 | | H1N1 | |
|---|---|---|---|---|---|---|---|
| | | | | EC₅₀ | H5N1 | EC50 | |
| | | | Molecu1ar | (@ 50% | EC₅₀ | (@ 50% | IC₅₀ |
| SRI # | Class | Structure | Weight | Via) | (Inf Pt) | Via) | (toxicity) |
| 22081 | 1a | 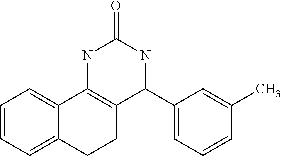 | 290.36811 | 1.5 μM 0.7 μM 0.91 μM | NA 0.55 μM 0.52 μM | | >50 μM |
| 22082 | 1a | 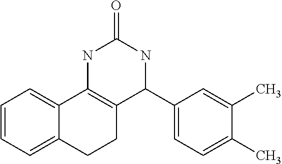 | 304.3952 | <0.8 μM 1.88 μM 0.59 μM | NA 0.93 μM 0.41 μM | | >50 μM |
| 22083 | 2a | 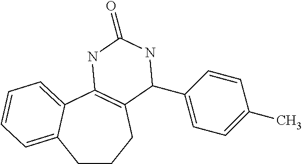 | 304.3952 | 28 μM | NA | 9.5 uM | >50 μM |
| 22084 | 2a | 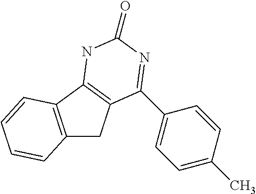 | 274.32508 | >50 μM | | | >50 μM |
| 22085 | 1a, 1b | 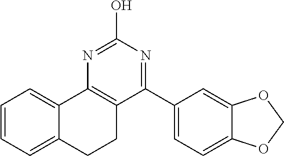 | 320 | <0.48 μM | NA | | >50 μM |
| | | 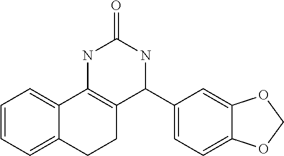 | | | | | |
| 22086 | 1a | 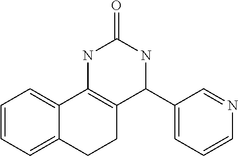 | 277.3286 | >50 μM | NA | | >50 μM |
| 22087 | 1a | 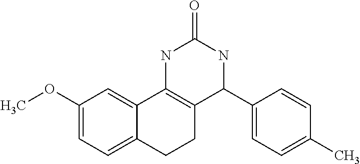 | 320.3946 | >50 μM | NA | 47 uM | >50 μM |
| 22088 | 1a | 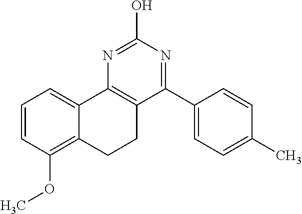 | 320 | 15.4 μM | | | >50 μM |
| | | 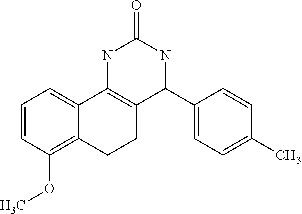 | | | | | |
| 22105 | 3a | 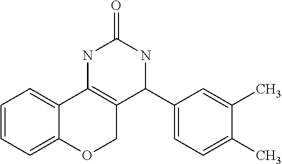 | 306.36751 | 1.8 μM 2.7 μM | 1.0 μM 1.9 μM | 14.9 uM | >50 μM |
| 22106 | 3b | 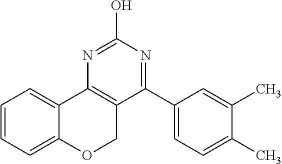 | 304.35157 | >50 μM | >50 μM | | >50 μM |
| 22107 | 3a | 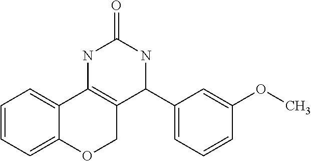 | 308.33982 | >50 μM | >50 μM | 46 uM | >50 μM |
| 22108 | 3b | 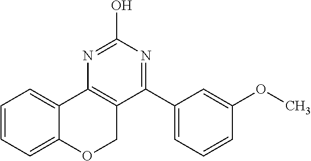 | 306.32388 | >50 μM | >50 μM | | >50 μM |
| 22109 | 3a | 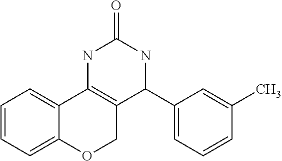 | 292.34042 | 2.8 μM 7.4 μM | 2.1 μM 5.1 μM | >50 μM | >50 μM |
| 22110 | 3b | 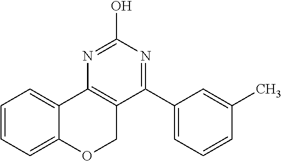 | 290.32448 | 10.4 μM | 8.6 μM | | >50 μM |
| 22111 | 1a | 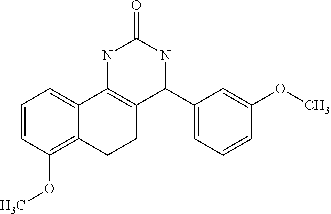 | 336.394 | >50 μM | >50 μM | | >50 μM |
| 22112 | 1b | 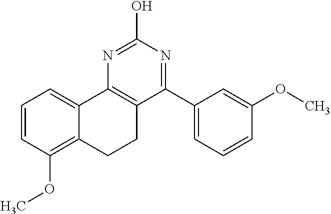 | 334.37806 | >50 μM | >50 μM | | >50 μM |
| 22113 | 1a | 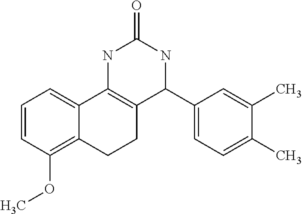 | 334.42169 | 2.5 μM 5.1 μM | 2.4 μM 4.4 μM | 16.8 uM | >50 μM |
| 22114 | 1b | 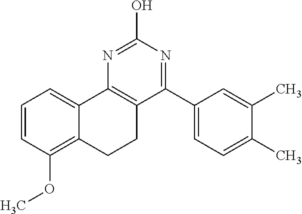 | 332.40575 | 13.5 μM 9.1 μM | 12.0 μM 0.70 μM | | >50 μM |
| 22115 | 1a | 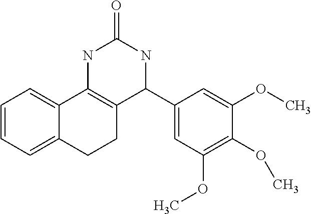 | 366.42049 | >50 μM | >50 μM | | >50 μM |
| 22116 | 1b | 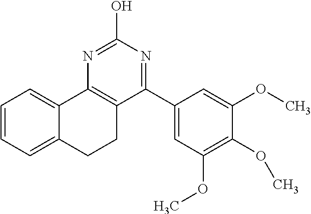 | 364.40455 | >50 μM | >50 μM | | >50 μM |
| 22117 | 2a | 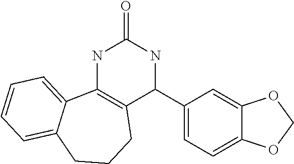 | 334.37806 | 4.8 μM 6.8 μM | 2.9 μM 4.0 μM | | >50 μM |
| 22118 | 2b | 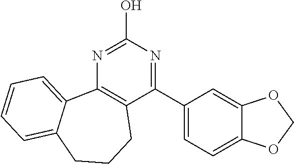 | 332.36212 | >50 μM | >50 μM | | >50 μM |
| 22119 | 1a | 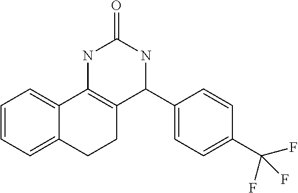 | 344.3394 | 32.9 μM | 14.8 μM | 11.7 uM | >50 μM |
| 22120 | 1a | 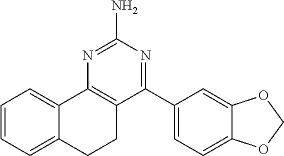 | 317.3503 | >50 μM | >50 μM | | >50 μM |
| 22121 | 1a | 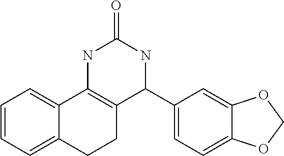 | 320.35097 | 0.39 μM NI | 0.34 μM 0.22 μM | >50 μM | 29.6 μM |
| 22122 | 1b | 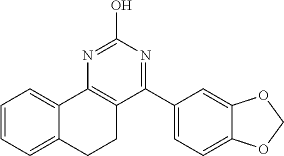 | 318.33503 | >50 μM | >50 μM | | >50 μM |
| 22123 | 1a | 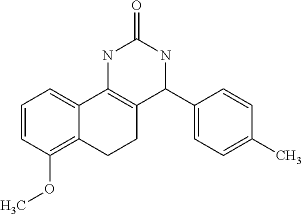 | 320.3946 | 18.3 μM | 10.9 μM | >50 μM | >50 μM |
| 22124 | 1b | 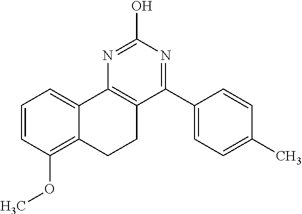 | 318.37866 | >50 μM | >50 μM | | >50 μM |
| 22125 | 1b | 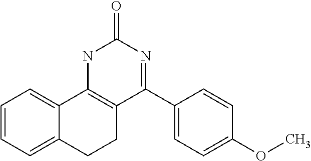 | 304.35157 | 0.96 μM 1.62 μM | 0.92 μM 1.45 μM | 24 uM | >50 μM |
| 22126 | 2a | 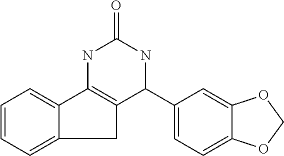 | 306.32388 | >50 μM | >50 μM | | >50 μM |
| 22127 | 2a | 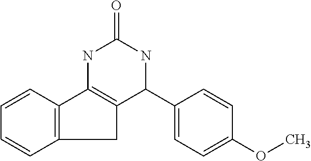 | 292.34042 | >50 μM | >50 μM | | >50 μM |
| 22128 | 2b | 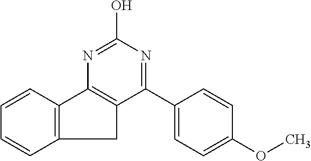 | 290.32448 | >50 μM | >50 μM | | >50 μM |
| 22129 | 3a | 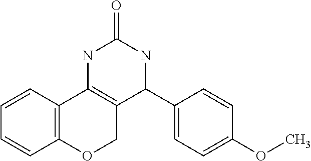 | 308.33982 | >50 μM | >50 μM | | >50 μM |
| 22130 | 1a | 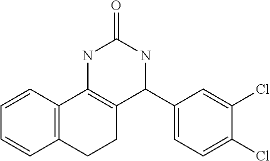 | 345.23108 | 1.7 μM 0.097 μM | 1.4 μM 0.088 μM | | >50 μM |
| 22342 | 1a | 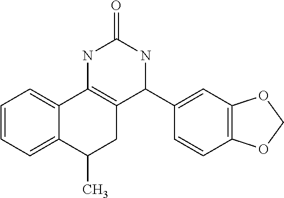 | 334.37806 | 0.72 μM | 0.39 μM | | >50 μM |
| 22343 | 1a | 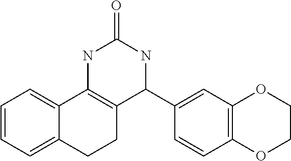 | 334.37806 | 45.7 μM | 31.9 μM | | >50 μM |
| 22344 | 1a | 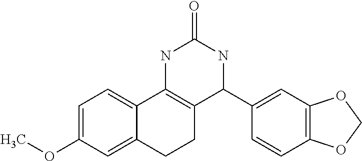 | 350.37746 | >50 μM | >50 μM | | >50 μM |
| 22345 | 2a | 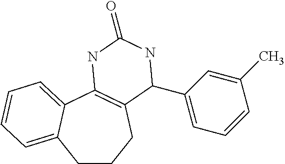 | 304.3952 | 18.8 μM | 13.6 μM | | >50 μM |
| 22346 | 1a | 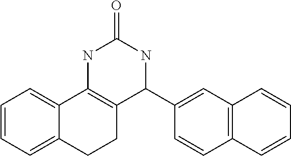 | 326.40156 | 55 μM | >50 μM | | >50 μM |
| 22347 | 1a | 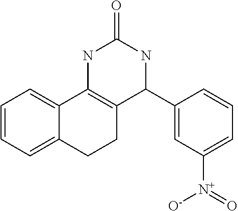 | 321.33855 | 5.5 μM | 4.2 μM | >50 μM | >50 μM |
| 22348 | 1a | 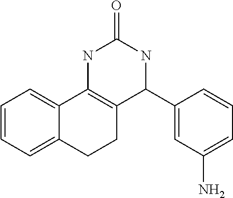 | 291.35569 | >50 μM | >50 μM | | >50 μM |
| 22349 | 1a | 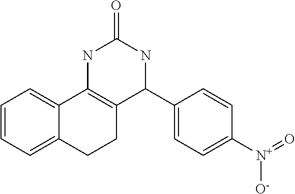 | 321.33855 | 1.17 μM | 0.95 μM | 17.7 uM | >50 μM |
| 22350 | 1a | 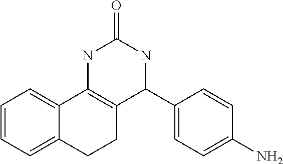 | 291.35569 | 30.6 μM | 17.9 μM | | >50 μM |
| 22402 | 2a | 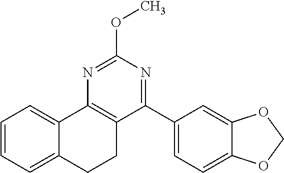 | 332.36212 | 8.4 μM 14.5 μM | 5.2 μM 5.7 μM | | >50 μM |
| 22403 | 2a | 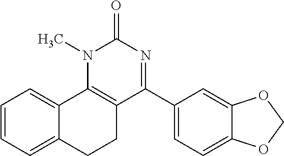 | 332.36212 | 5.2 μM 5.7 μM | 1.7 μM 2.0 μM | | >50 μM |
| 22404 | 3a | 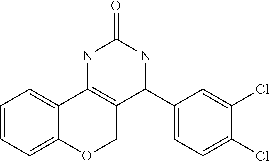 | 347.20339 | 1.6 μM 0.94 μM | 1.4 μM 0.65 μM | >50 μM | >50 μM |
| 22405 | 1a | 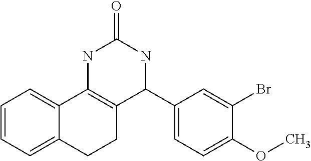 | 385.26354 | 51 nM NI 15 nM 4.7 nM 25 nM | 42 nM 38 nM 13.8 nM 6.2 nM 26 nM | 425 nM | >50 μM |
| 22406 | 1a | 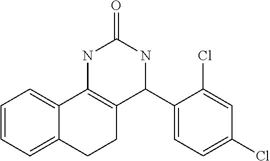 | 345.23108 | >50 μM | >50 μM | >50 μM | >50 μM |
| 22407 | 1a | 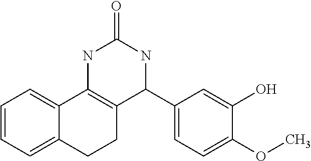 | 322.36691 | >50 μM | >50 μM | | >50 μM |
| 22408 | 1a | 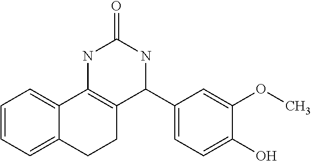 | 322.36691 | >50 μM | >50 μM | | >50 μM |
| 22409 | 1a | 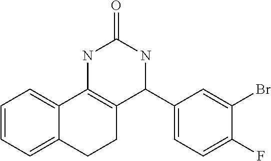 | 373.22748 | 140 nM 52 nM 27.5 nM 20 nM | 99 nM 37.5 nM 27 nM 20 nM | 1.4 uM | >50 μM |
| 22410 | 1a | 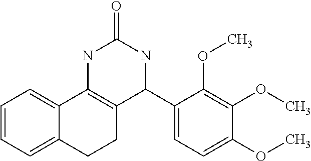 | 366.42049 | >50 μM | >50 μM | | >50 μM |
| 22411 | 1a | 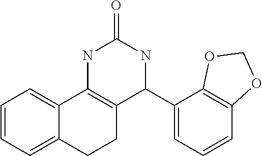 | 320.35097 | >50 μM | >50 μM | | >50 μM |
| 22412 | 2a | 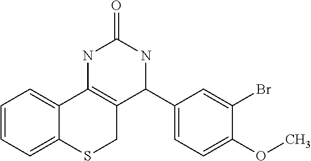 | 403.30045 | 139 nM 53 nM 34 nM 17 nM | 78 nM 41 nM 23 nM 17 nM | 351 nM | >50 μM |
| 22421 | 3a | 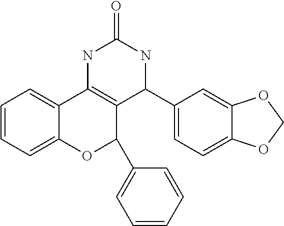 | 398.42206 | >50 μM | >50 μM | >50 μM | >50 μM |
| 22422 | 3b | 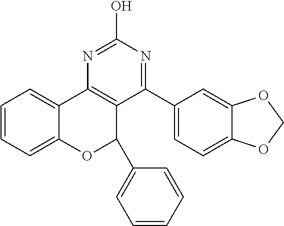 | 396.40612 | >50 μM | >50 μM | | >50 μM |
| 22516 | 1a | 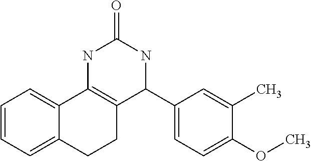 | 320.3946 | 75 nM 42 nM 36 nM 57 nM | 54 nM 32 nM 27 nM 1.7 μM | 10 uM | >50 μM |
| 22517 | 1a | 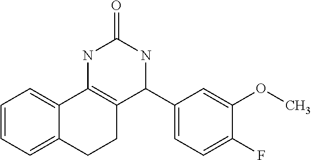 | 324.35794 | 9.6 μM | 7.2 μM | >50 μM | >50 μM |
| 22518 | 1a | 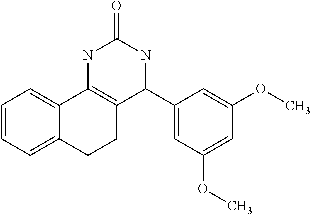 | 336.394 | 4.7 μM | 3.4 μM | | >50 μM |
| 22519 | 3a | 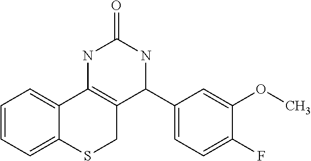 | 342.39485 | NI | 6.5 μM | | >50 μM |
| 22520 | 1a | 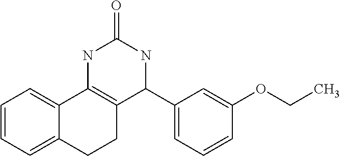 | 320.3946 | NI | 7.1 μM | >50 μM | >50 μM |
| 22521 | 3a | 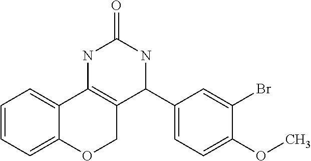 | 387.23585 | 77 nM 93 nM 64 nM 6 nM | 56 nM 79 nM 66 nM 6 nM | 4.0 uM | >50 μM |
| 22522 | 1a | 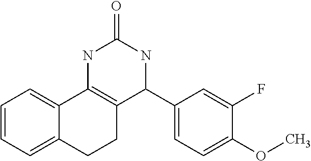 | 324.35794 | 0.26 μM | 0.19 μM | 3.8 uM | >50 μM |
| 22523 | 3a | 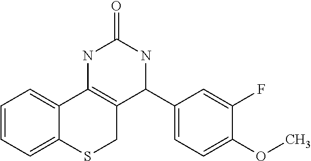 | 342.39485 | 0.76 μM | 0.55 μM | | >50 μM |
| 22524 | 1a | 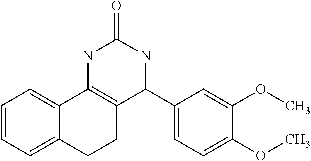 | 336.394 | 7.4 μM* | 7.0 μM | 39 uM | >50 μM |
| 22526 | 1a | 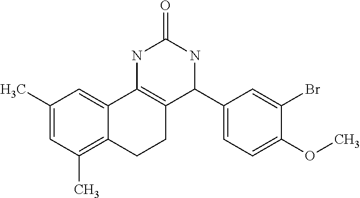 | 413.31772 | >48 μM | >48 μM | >50 μM | 6.9 μM |
| 22700 | 1a | 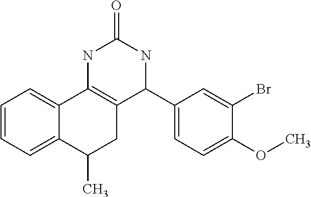 | 399.29063 | 52 nM 13.6 nM 9 nM | 40 nM 11 nM 9 nM | 98.5 nM | >48 μM |
| 22701 | 3a | 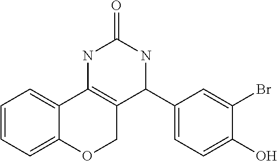 | 373.20876 | 26.2 nM 27 nM | 21 nM 27 nM | 1.6 uM | >20 μM |
| 22702 | 1a | 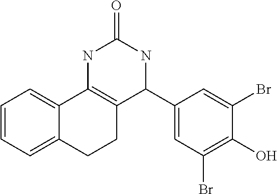 | 450.13248 | >40 μM | >40 μM | | >40 μM |
| 22703 | 1a | 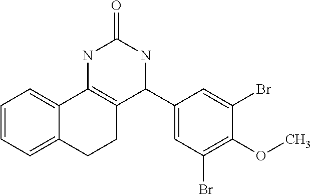 | 464.15957 | 38 μM | >40 μM | >40 μM | >40 μM |
| 22704 | 1a | 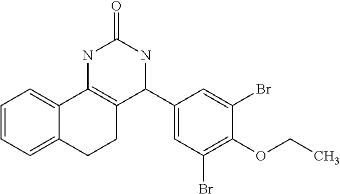 | 478.18666 | >40 μM | >40 μM | >40 μM | >40 μM |
| 22705 | 1a | 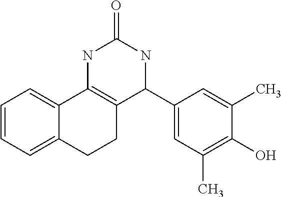 | 320.3946 | >40 μM | 10 μM | | >40 μM |
| 22706 | 1a | 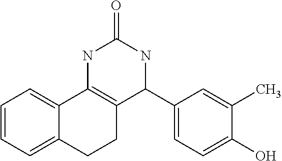 | 306.36751 | 137 nM NI 57 nM | 127 nM NI 8.558 μM | 4.8 uM | >50 μM |
| 22707 | 1a | 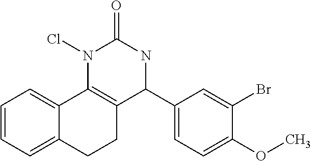 | 419.70857 | 192 nM | 186 nM | | >50 μM |
| 22708 | 1a | 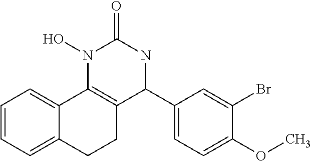 | 401.26294 | 1.1 μM | >0.95 μM | | >50 μM |
| 22709 | 1a | 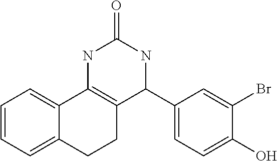 | 371.23645 | 18.6 nM 5.0 nM 69 nM | 18.0 nM 4.6 nM 69 nM | 118 nM | >53 μM |
| 22710 | 1b | 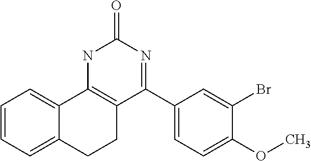 | | >50 μM | >50 μM | | >50 μM |
| 22711 | 1a | 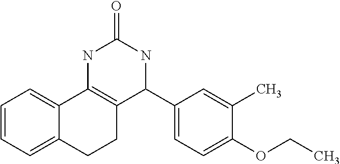 | 334.42169 | 1.3 μM | 1.2 μM | 37 uM | >50 μM |
| 22722 | 1a | 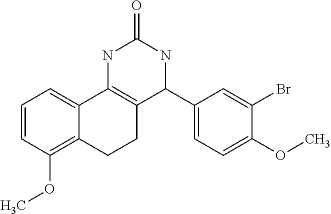 | 415.29003 | >62 nm | >62 nm | 1.1 uM | >62 nm |
| 23096 | 2a | 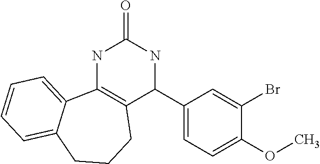 | 399.29063 | 242 nM | 280 nM | 153 nM | >40 μM |
| 23097 | 3a | 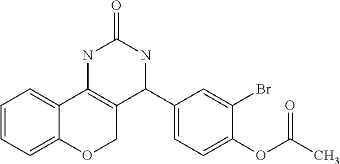 | 415.2464 | 92.8 nM | 102 nM | 1.0 uM | >40 μM |
| 23098 | 3a | 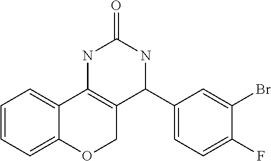 | 375.19979 | 1.2 μM | 1.3 μM | >40 μM | >40 μM |
| 23099 | 3a | 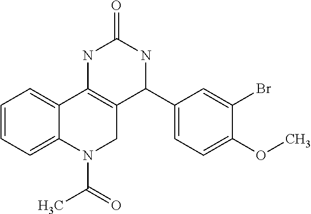 | 428.28876 | 1.2 nM 6.4 nM | 1.4 nM 6.0 nM | 6.2 nM | >40 μM |
| 23100 | 3a | 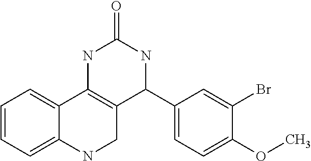 | 386.25112 | 637 nM | 718 nM | | >40 μM |
| 23101 | 3a | 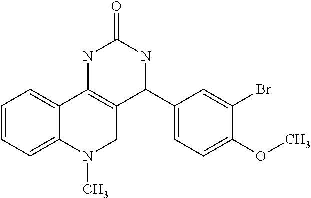 | 400.27821 | >6 μM | >6 μM | | >40 μM |
| 23102 | 3a | 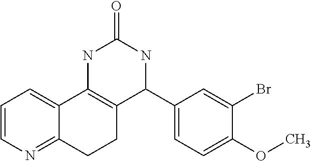 | 386.25112 | 14.5 μM | | >40 μM | >40 μM |
| 23205 | 3a | 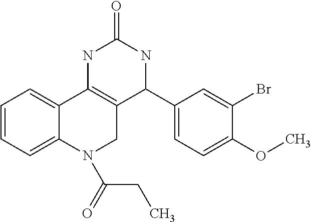 | 442.31585 | 193 nM | 189 nM | 10.1 uM | >40 μM |
| 23206 | 3a | 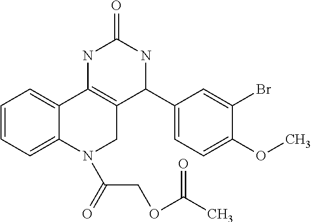 | 486.3258 | 22 nM 11.9 nM | 22 nM 11.2 nM | 25.7 nM | >40 μM |
| 23207 | 3a | 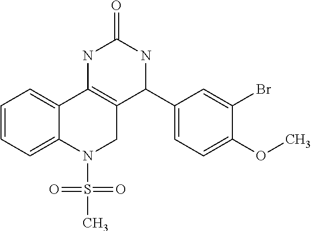 | 464.34101 | 29 μM | 31 μM | | >40 μM |
| 23208 | 3a | 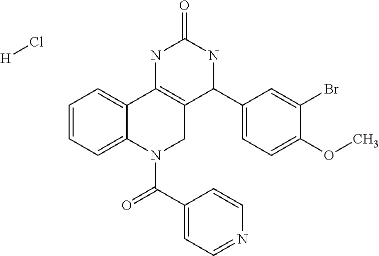 | 527.809 | >40 μM | >40 μM | | >40 μM |
| 23209 | 3a | 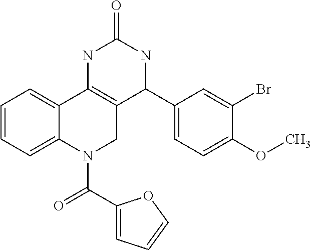 | 480.32161 | >40 μM | >40 μM | >40 μM | >40 μM |
| 23210 | 3a | 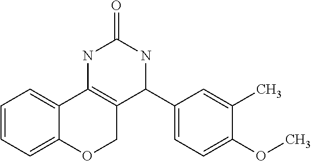 | 322.36691 | 332 nM | 307 nM | 3.1 uM | >40 μM |
| 23272 | 3a | 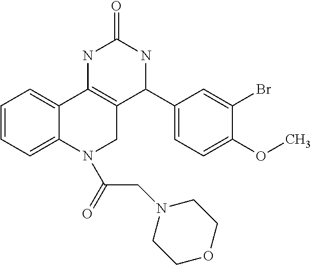 | 513.39525 | 9.1 μM | 8.7 μM | 3.5 uM | >40 μM |
| 23273 | 3b | 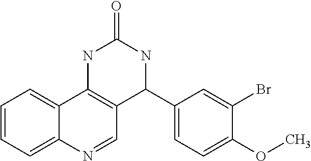 | 384.23518 | 2.1 μM | 1.6 μM | | >40 μM |
| 23274 | 3a | 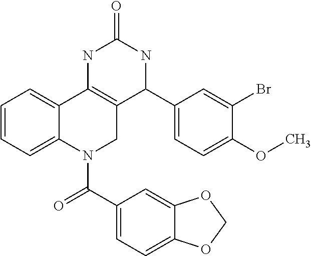 | 534.3704 | >40 μM | >40 μM | >40 μM | >40 μM |
| 23275 | 3a | 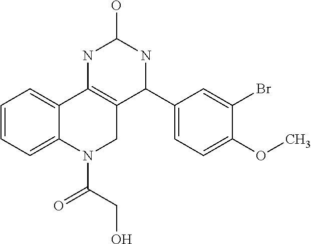 | 444.28816 | 2.7 nM | 2.7 nM | <67 nM | >40 μM |
| 24716 | 3a | 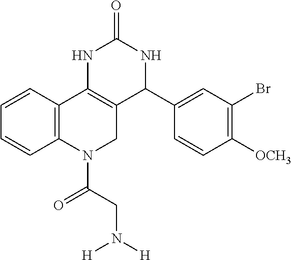 | 442.06 | 3.1 μM | 3.1 μM | | >40 μM |
| 24717 | 3a | 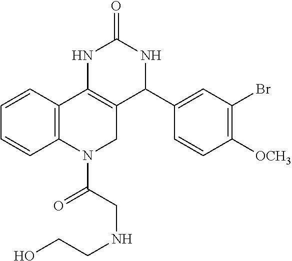 | 486.09 | 2.1 μM | 2.1 μM | 5.6 uM | >40 μM |
| 24718 | 3a | 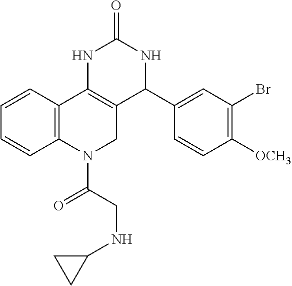 | 482.1 | 9.7 μM | 9.7 μM | 8.0 uM | >40 μM |
| 24719 | 3a | 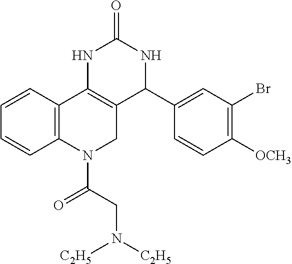 | 498.13 | 1.2 μM | 1.2 μM | | >40 μM |
| 25009 | 3a | 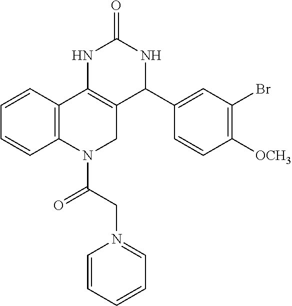 | 505.1 | 7.1 μM | 7.1 μM | | >40 μM |
| 25010 | 3a | 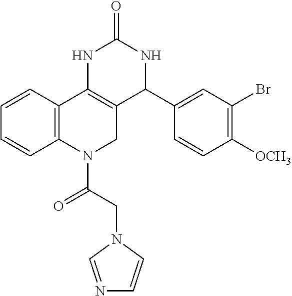 | 493.1 | <316 nM | 82 nM | | >40 μM |
| 25011 | 3a | 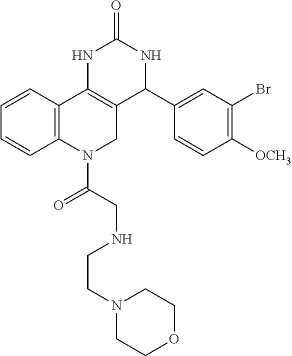 | 555.15 | 3.9 μM | 3.9 μM | | >40 μM |
| 25012 | 3a | 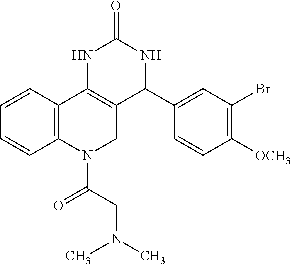 | 470.1 | <330 nM | 200 nM | 3.0 uM | >40 μM |
| 25350 | 3a | 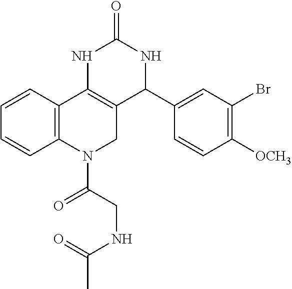 | 485.3 | 45 nm | 44 nM | 622 nM | >40 μM |
| 25408 | 3a | 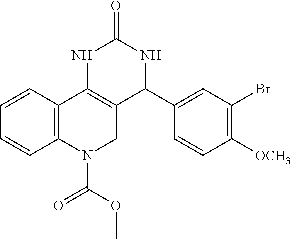 | 444.3 | 208 nM | 201 nM | 9.7 uM | >40 μM |
| 25410 | | 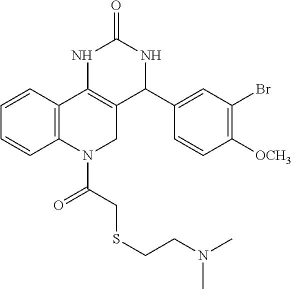 | 531.5 | 714 nM | 669 nM | 15.6 uM | |
| 25411 | | 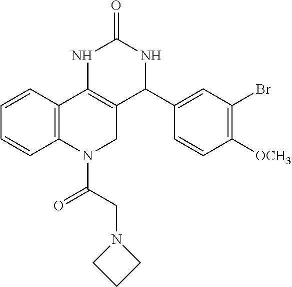 | 483.36 | 814 nM | 755 nM | 3.7 uM | >40 μM |
| 25412 | | 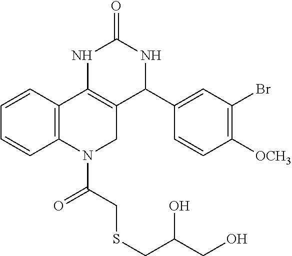 | 534.42 | 893 nM | 848 nM | 3.1 uM | >40 μM |
| 25413 | | 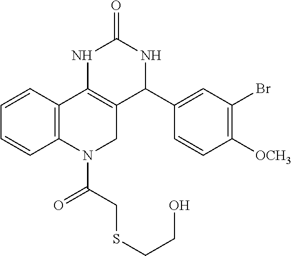 | 504.39 | 4.4 uM | 4.5 uM | 3.8 uM | >40 μM |
| 25414 | | 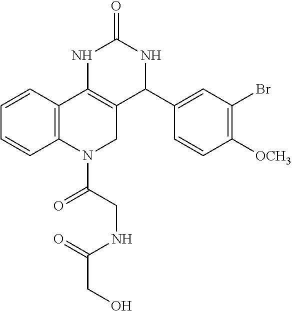 | 501.33 | 277 nM | 275 nM | >40 μM | >40 μM |
| 25484 | | 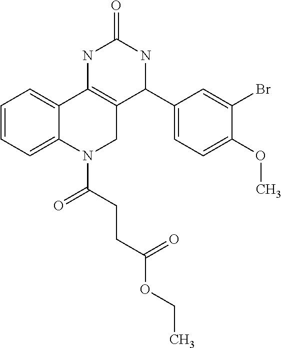 | 514.38 | >20 μM | | | |
| 25575 | | 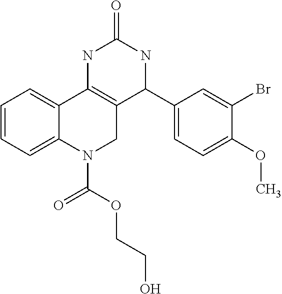 | 474.31 | 4.7 μM | | | |
| 25576 | | 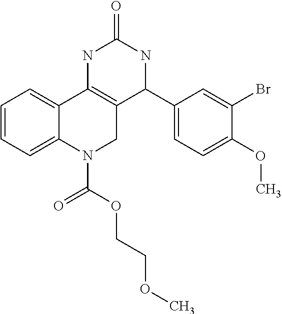 | 488.34 | >20 μM | | | |
| 25577 | | 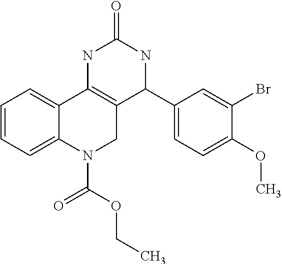 | 458.32 | >20 μM | >20 μM | | |
| 25578 | | 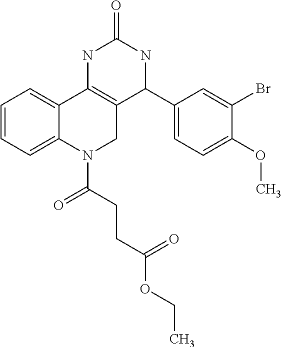 | 514.38 | >20 μM | >10 μM | | |
| 28000A | | 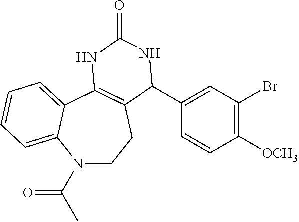 | 442.31 | >20 μM | | | |
| 28001A | | 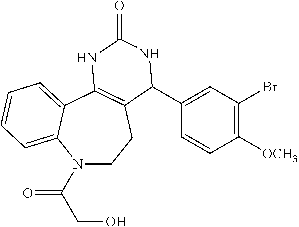 | 458.31 | >20 μM | | | |
| 28002A | | 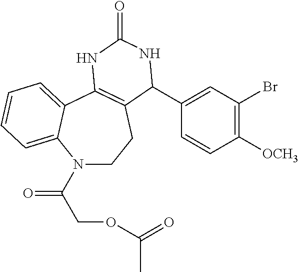 | 500.34 | >20 μM | | | |
| 28003A | | 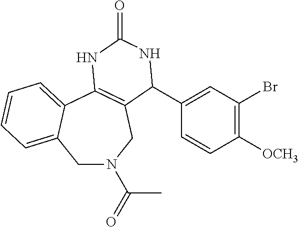 | 442.31 | | | | |
| 28004A | | 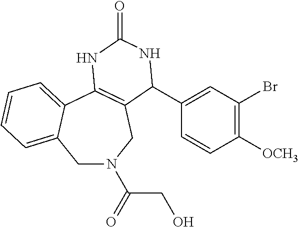 | 458.31 | | | | |
| 28005A | | 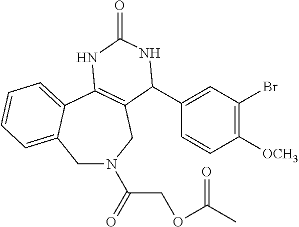 | 500.34 | | | | |
| 28006A | | 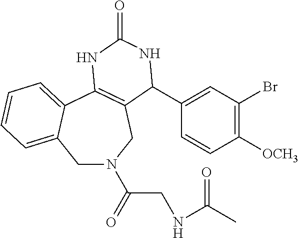 | 499.36 | | | | |
| 28007A | | 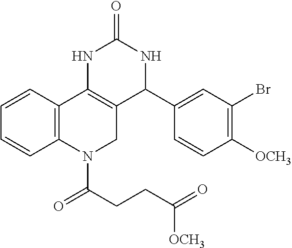 | 500.34 | | | | |
| 28008A | | 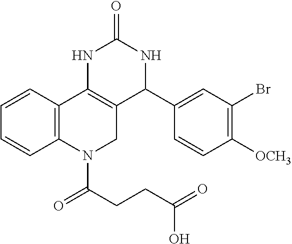 | 486.32 | | | | |
| 28009A | | 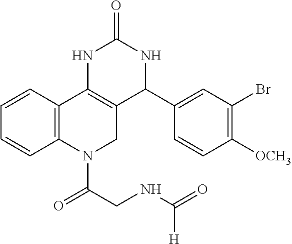 | 471.31 | | | | |
| 28010A | | 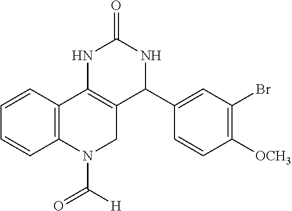 | 414.25 | | | | |
| 28011A | | 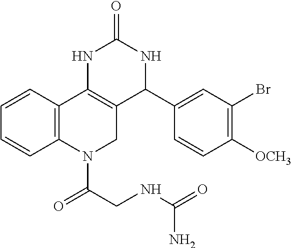 | 486.32 | | | | |

### pK Evaluation of Eight Compounds with Activity Against Influenza a Viruses

The peak plasma concentration and the half-life of selected compounds were evaluated for in vivo activity. HPLC procedures were developed to measure the concentration of eight compounds in mouse plasma. The agents eluted from a reverse phase HPLC column (5 μm BDS Hypersil C-18 column, 150×4.6 mm, ThermoHypersil-Keystone Scientific Inc., Bellfonte, Pa.) using a 25, 30, or 50% acetonitrile in 50 mM ammonium dihydrogen phosphate buffer as the mobile phase. The compounds were detected as they eluted from the column by their UV absorbance at 260 nm.

Three mice were sacrificed 15, 30, and 60 minutes after the injection (IP) of 100 mg/kg SR 22521, SR 23096, SR 23206, SR 23099, SR 25010, or SR 25350. With SR 23275 and SR 25012 there were 4 mice per treatment group, a survival bleed was performed to get two blood samples per mouse, and samples were taken 5, 15, 30, and 60 after the injection of 100 mg/kg of compound. Mice blood was collected in heparinized microcapillary tubes containing lithium heparin. Blood was kept at room temperature and centrifuged at 2,400 rpm for 15 min at room temperature (23° C.) within 1 hour of blood collection. The plasma samples were filtered through a centrifugation filter device (Centrifree, AMicon) to remove plasma proteins and to prepare the sample for HPLC analysis.

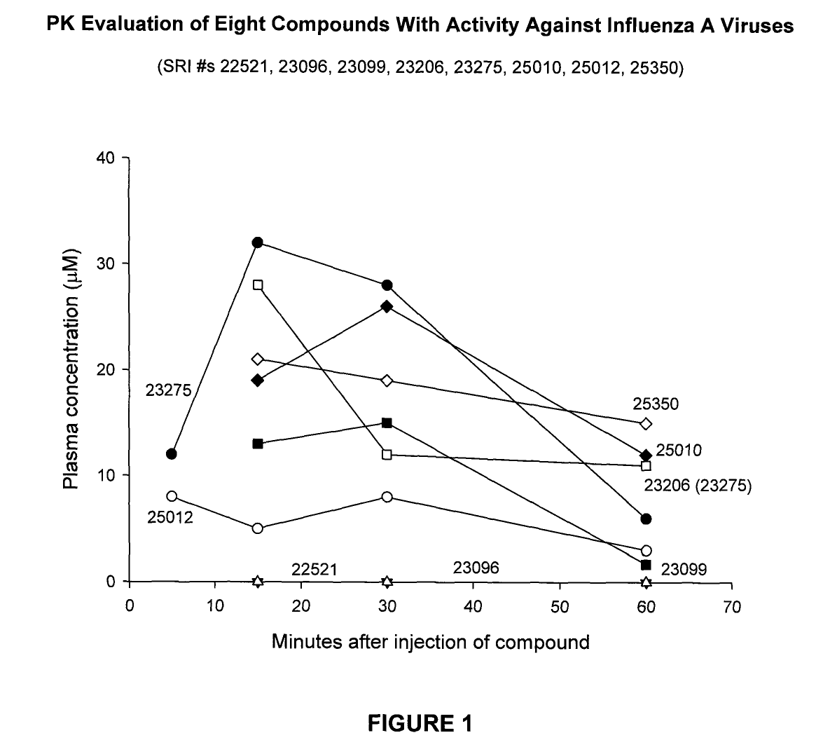

Plasma samples were analyzed using HPLC as described above. The data is presented graphically in FIG. 1. Each value in FIG. 1 represents the mean±standard deviation from three or four samples. These results indicate that significant plasma levels (μM) were achieved with five of the eight compounds and that these compounds were retained in the plasma for over 1 hour.

### General Procedure for Synthesis of Compounds

The following general procedures should enable anyone skilled in the art of synthesis to prepare these classes of compounds once aware of the present disclosure. Several specific examples follow the general methods.

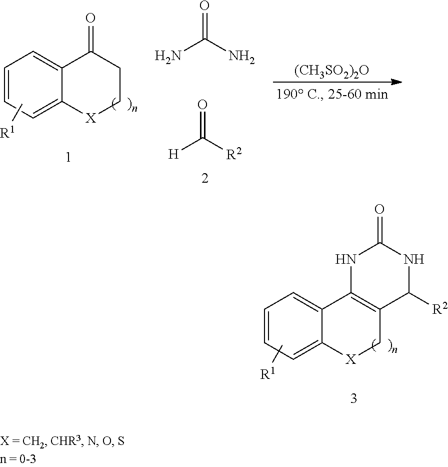

Method A

A ketone (represented in the Scheme by 1 or a similar ketone) (2.0 mmole), urea (180 mg), an aldehyde (2, 2.0 mmole) and methylsulfonic anhydride (150 mg) in 2 ml of anhydrous CH₃CN were placed in a 8 ml reaction vessel and treated with microwave at 200 W, 190-200° C. for 25 min. To the cooled reaction mixture 2 ml of ethanol was added. The mixture was treated with microwave at 120 W, 120° C. for 2 min. The reaction mixture was cooled and the solid was collected by filtration, washed with water, acetone, then suspended in 2 ml of chloroform. The suspension was treated with microwave at 120 W, 120° C. for 2 min, cooled in freezer, and then filtered to give 3 as a colorless solid.

For compounds soluble in ethanol or chloroform, passage through a chromatography column is used to purify the final compounds.

Method B

In a 50 mL round-bottomed flask equipped with a condenser, a ketone (represented in the Scheme by 1 or a similar ketone) (20.0 mmole), an aldehyde (2, 20.0 mmole), urea (1.80 g) and methylsulfonic anhydride (0.5 g) in 20 mL of DMSO (anhydrous) was heated to 190-210° C. in an atmosphere of argon for 1 h. To the cooled solution 60 mL of ethanol was added and the mixture was put in refrigerator for 30 min. The precipitate was collected by filtration, washed with water, acetone, then suspended in 50 mL of chloroform, stirred at 200° C. for 25 min in a steel bomb, then cooled to rt. The precipitate 3 was collected by filtration to give a colorless solid, typically 2 to 3 gram.

For compounds soluble in ethanol or chloroform, a column is often needed to purify the product.

### 1. Synthesis of SRI 22405

Method B. 3.75 g.

### 2. Synthesis of SRI 22409

Method A. 358 mg.

### 3. Synthesis of SRI 22412

Method B. 3.12 g

### 4. Synthesis of SRI 22516

Method A. 208 mg.

### 5. Synthesis of SRI 22521

Method B. 2.2 g.

### 6. Synthesis of SRI 22700

Method B. 2.65 g

### 7. Synthesis of SRI 22701

In a 50 mL round-bottomed flask equipped with a condenser, a 4-chromanone (2.96 g, 20 mmole), 3-bromo-4-hydroxy-benzaldehyde (20.0 mmole), urea (1.80 g) and methylsulfonic anhydride (0.45 g) in 15 mL of DMSO (anhydrous) was heated to 180-190° C. in an atmosphere of argon for 45 min. To the cooled reaction mixture acetic anhydride (5.0 mL), triethylamine (6.0 mL) was added. The solution was stirred for 25 min, then transferred into a beaker containing ice-water (120 g), Na₂CO₃ (5 g) and Na₂SO₄ (5 g). The solid (gum) was collected by filtration, washed with water. The mother liquor was extracted with ethyl acetate twice. The organic layer was combined with the solid, evaporated, treated with acetone in a sonicator for 15 min. large quantities of precipitate formed. The mixture was stored in a freezer overnight. The precipitate was collected by filtration, washed acetone to give a colorless solid (2.69 g), which was treated with 2M NH₃ in ethanol for 1 hour. The solvents were removed and the residue was treated ethanol (25 mL) and acetic acid (3 mL) at room temperature for 1 hour. The precipitate was collected by filtration. SRI 22701 was obtained as a colorless solid (2.1 g).

### 8. Synthesis of SRI 23099

2,3-Dihydro-1H-quinolin-4-one (3.3 g) was treated with acetic anhydride (3.3 mL) in pyridine for 1 hour. The solution was concentrated on a rotovapor. The residue dissolved in 50 mL of chloroform, washed with brine, and water. The solvents were evaporated again. The residue was dissolved in acetone, ether, and hexane, and chloroform, stored in freezer overnight. The precipitated was collected by filtration to give 2,3-Dihydro-1-acetyl-quinolin-4-one as a colorless solid (3.16 g), which is used for the synthesis of SRI 23099 without further purification.

SRI 23099 was synthesized with method A (109 mg) as a colorless solid.

### 10. Synthesis of SRI 23207

2,3-Dihydro-1H-quinolin-4-one (0.70 g) was treated with methylsulfonic chloride (1.2 eq) in pyridine (5 mL) overnight at room temperature. The solution was concentrated on a rotovapor. The residue was dissolved in chloroform, purified on a column, recrystallized from acetone, ether, and hexane to give 2,3-Dihydro-1-methylsulfonyl-quinolin-4-one as a colorless solid (370 mg), which is used for the next step without further purification. SRI 23207 was synthesized with method A (358 mg) as a colorless solid.

### 11. Synthesis of SRI 23206

2,3-Dihydro-1H-quinolin-4-one (0.70 g) was treated with acetoacetyl chloride (1.2 eq) in pyridine (5 mL) for 1 hour at room temperature. The reaction was quenched by adding 1 mL of ethanol. The solution was concentrated on a rotovapor. The residue was dissolved in chloroform, purified on a column, recrystallized from acetone, ether, and hexane to give 2,3-Dihydro-1-N-acetoacetyl-quinolin-4-one as a colorless solid (470 mg), which is used for the next step without further purification. SRI 23206 was synthesized with method A, purified with a column to give the titled compound (180 mg) as a colorless solid.

### 12. Synthesis of SRI 28010A

2,3-Dihydro-1H-quinolin-4-one (1.0 g) was dissolved in pyridine (4.0 mL). 2.0 mL of acetic formic anhydride (10 mL of formic acid and 8.0 mL of acetic anhydride refluxed for 1 h) was added. The mixture was stirred at 100° C. overnight. The solution was concentrated, and the residue was purified on a column, recrystallized from acetone/ether/hexane to give a colorless solid (0.69 g). Follow the procedure of exp. 8. SRI 28010A was obtained as colorless solid (490 mg).

### 13. Synthesis of SRI 23209

2,3-Dihydro-1H-quinolin-4-one (0.50 g) was treated with 2-furoyl chloride (1.2 eq) in chloroform (10 mL) and triethylamine (1.2 mL) for 1.5 hour at room temperature. The reaction was quenched by adding 0.5 mL of water. The solution was concentrated and the residue was dissolved in chloroform (25 mL), washed with water (5 mL×2). The solvents was evaporated and the residue was dissolved in acetone and ethyl ether, stored in freezer overnight. 2,3-Dihydro-1-N-furoyl-quinolin-4-one was obtained as a colorless solid (470 mg), which is used for the next step without further purification. SRI 23209 was synthesized with method A (360 mg) as a colorless solid.

### 14. Synthesis of SRI 23098

Method B. 0.69 g (starts from 1.2 g of 4-chromanon).

### 15. Synthesis of SRI 23096

Method B. 1.42 g (starts from 1.2 g of 1-benzosuberone).

### 16. Synthesis of SRI 23102

Method A. 36 mg (starts from 180 mg).

### 17. Synthesis of SRI 22274

Method A. 484 mg.

### 18. Synthesis of SRI 23273

SRI 23099 (200 mg) was treated with NaOH (0.1 g) in ethanol (10 mL) and water (1.0 mL) at 60° C. for 1 h. TLC suggested that the starting martial is gone. The reaction was quenched with 0.3 mL of acetic acid, and the mixture was concentrated. The residue was purified on a column. the major fraction was collected and precipitated from acetone to give the titled compound as a colorless solid (54 mg). MS and NMR suggested that it is the oxidized compound SRI 23273.

### 19. Synthesis of SRI 23275

2,3-Dihydro-1H-quinolin-4-one (5.8 g) was dissolved in pyridine (10 mL) and chloroform (100 mL) at 0° C. To the solution 2-acetoxyacetyl chloride (6.0 mL) was added dropwisely. The solution was stirred for 1 hour. Ethanol (5 mL) was added to quench the reaction. The solution was concentrated on a rotovapor. The residue dissolved in 150 mL of chloroform, washed with brine, and water. The solvents were evaporated again. The residue was purified with a column. The major fraction was collected, recrystallized from chloroform/hexane, then acetone/hexane to give a colorless solid (6.85 g), which was used for the next step of synthesis without further purification.

The above solid (6.0 g), 3-bromo-4-methoxybenzaldehyde (5.4 g) and urea (2.7 g) was dissolved in DMSO (30 mL). To the solution 0.8 g of methyl sulfonic anhydride was added. The mixture was stirred at 180-185° C. for 3 h, cooled, poured into a beaker containing 50 mL of cold water. The gum was collected, treated with 150 mL 7N ammonia in methanol at room temperature for 24 hours. Large amount of precipitate can be observed. The mixture was put in a freezer for 1 h, filtered. The solid washed ethanol and acetone then treated with 7N ammonia in methanol again over night to give SRI 23275 as a colorless solid (5.10 g).

### 20. Synthesis of SRI 25010

SRI 23275 (2 g) was dissolved in 10 mL of pyridine. To the solution 20 mL of CH₃CN was added. The solution was cooled in an ice bath. Methylsulfonic chloride (3 mL) was added dropwisely within 20 min. The solution was stirred for another 25 minutes, then poured into a beaker containing 200 mL of cold ethyl ether. The precipitate was dissolved in cold acetone/ethanol. The acetone was evaporated on a rotovapor. To the remaining solution 200 mL of cold water was poured slowly. The precipitates was collected by filtration, dried in vacuum to give a colorless solid (A, 1.68 g) which is used for next step of synthesis without further purification.

The above solid a (150 mg) was suspended in ethanol (10 mL). Imidazole (0.3 g) was added. The suspension was stirred at 80° C. for 1 h to give a clear solution. The solvent was removed and the residue was purified with a column. The major fraction was collected, recrystallized from acetone/ethyl ether to give SRI 25010 as a colorless solid (74 mg).

### 21. Synthesis of SRI 25350

Solid A in exp. 19 (350 mg) was suspend in 30 mL of ammonia in 2-propanol (saturated at 0° C.) and stirred at room temperature for 36 hours in a sealed round-bottom flask. The solvent was removed. The residue was treated with ethyl ether. The precipitated was collected by filtration to give a colorless solid (330 mg), which was treated with acetic anhydride in pyridine for 45 min. the solvent was removed, and the residue purified with a column to give SRI 25350 as a colorless solid (165 mg).

### 22. Synthesis of SRI 28009A

The compound was synthesized in a similar procedure as SRI 25350 except that acetic formic anhydride was used.

### EXPERIMENTAL PROTOCOLS

A cytopathogenic effect (CPE) based assay to screen large compound libraries (>100,000 compounds) against influenza strain A/VN/1203/2004 (H5N1) has been developed according to this disclosure. The assay measures influenza-induced CPE in Madin Darby Canine Kidney (MDCK) cells using cell viability as the end point. Cell viability was determined from intracellular ATP concentration using firefly luciferase as the reporter. Percent cell viability was calculated by using mean luminescence values of the virus-infected cells in the presence of compound divided by the uninfected cell control×100.

Cell Culture: MDCK cells (ATCC CCL-34, American Tissue Culture Type) were maintained as adherent cell lines in Eagle minimum essential medium with 2 mM L-glutamine and 10% fetal bovine serum (FBS) at 37° C. in a humidified 5% CO₂ atmosphere as described previously [4]. Cells were passaged as needed and harvested from flasks using 0.25% trypsin-EDTA. Prior to cell plating, cells were resuspended in serum-free DMEM with 4 mM L-glutamine and 1% BSA (Assay Media).

Influenza virus culture: Influenza virus strain A/VN/1203/2004 was generated using a reverse genetics system and amplified in MDCK cells. The supernatant from transfected MDCK cells was used to infect a fresh MDCK cell field, and a single plaque was selected and resuspened in serum-free Dulbecco's modified Eagle's medium (DMEM, Invitrogen, Carlsbad, Calif.) containing 1% bovine sebum albumin (BSA, Invitrogen 15260-037, Fraction V) [5]. The plaque purified virus was used to inoculate 10-day old embryonated chicken eggs (SPF grade, Charles River Laboratories, Wilmington, Mass.). After inoculation, infected eggs were incubated for an additional 2 days and then placed at 4° C. overnight to terminate the embryo. The following day the egg allantoic fluid was recovered by (5-10 mL/egg), solid debris was removed by centrifugation, and aliquoted and stored below −80° C. for use in the assay. Viral titer and multiplicity of infection (MOI) values were established by plaque assays. The virus stocks from the allantoic fluid cells were titrated in MDCK cells using the TCID₅₀ method. The final titer was at 1×10⁷ TCID₅₀/ml (TCID₅₀. 50% tissue culture infectious dose).

Influenza virus strain A/CA/04/2009 (H1N1) was obtained from the CDC (CDC#2009712047; SR PASS E2; 052909CAL0409) and used to inoculate chicken eggs for amplification as previously described. Egg allantoic fluid was recovered, aliquoted and stored below −80° C. for use in the assay. Viral titer and multiplicity of infection (MOI) values were established by plaque assays. The virus stocks from the allantoic fluid cells were titrated in MDCK cells using the TCID₅₀ method. The final titer was at 1×10⁶ TCID₅₀/ml (TCID₅₀: 50% tissue culture infectious dose).

Cell Plating: 15,000 cells/well were plated in 96 well black clear-bottom tissue culture treated plates in 50 uL using a Matrix WellMate. The assay plates were incubated overnight at 37° C., 5% CO₂ and high humidity.

Control Drug and Test Compound Dose Response Format: The positive control drug for this assay, ribavirin [6] (#196066, MP Biomedicals, Solon, Ohio) was solubilized at 8 mg/ml in dimethyl sulfoxide (DMSO; Sigma, St. Louis, Mo.). The stock solution was diluted to final concentration of 164 μM in assay media (DMEM without phenol red, 1.0% BSA, 4 mM L-glutamine, 100 U/mL penicillin and 100 μg/ml streptomycin; Gibco, Grand Island, N.Y.) before each experiment and discarded afterwards. After 24 hrs, control drugs were diluted in assay media and added to each plate at a final concentration of 164 uM for Ribavirin (positive control). DMSO concentration of 0.5% was maintained for all control wells. Test compounds were evaluated by measuring their antiviral activity, cell toxicity, and selectivity. This involved carrying out dose-response curves over a 256-fold concentration range for cytotoxicity (uninfected cells) and antiviral activity (H5N1-infected cells). The compound drugs were added to assay media by two-fold serial dilutions, and then added to the plate wells for a final well compound concentration ranging from 50 μM to 0.39 μM (for cytotoxicity) and 66 nM to 0.5 nM (for antiviral activity). 25 μl of each compound was added to each well containing cells. Final DMSO concentration in each well was 0.5%.

Virus Addition: The plates were then immediately transferred from HTS facilities to a class II Biosafety Cabinet within the BSL-3 laboratory. Twenty-five (25 ul) of diluted influenza A/VN/1203/2004 virus (100 TCID₅₀ doses), diluted from amplified virus stock in egg allantoic fluid into assay media, for a final virus stock dilution of 1:10,000 which corresponds to an MOI of 0.005 was added to compound wells and the virus control wells. Addition of influenza A/CA/04/2009 virus required the addition of L-1-tosylamino-2-phenylethyl chloromethyl ketone (TPCK) trypsin (Sigma, St. Louis, Mo.) 2.0 μg/mL final concentration into assay media containing 0.1% BSA. Media only (mock virus) was added to the cell control wells. All additions were done using a Matrix WellMate (Hudson, N.H.). The plates were incubated for 72 h within the BSL-3 laboratory at 37° C., 5% CO₂ and high humidity.

Endpoint Read: After incubation, the assay plates were equilibrated to room temperature for 30 min and an equal volume (100 μL) of CellTiter-Glo reagent (Promega Inc.) was added to each well using a WellMate (Matrix, Hudson, N.H.). Plates were shaken (in the BSC) for two minutes at speed 5 on a Labline Instruments (Kochi, India) plate shaker. Luminescence was then measured using a Perkin Elmer Envision™ multi-label reader (PerkinElmer, Wellesley, Mass.) with an integration time of 0.1 s. This step was also performed within the BSL-3 facility.

Data Analysis: Data was analyzed using ActivityBase software (IDBS, Inc, Guilford, UK). Eight control wells containing cells only and four wells containing cells and virus were included on each assay plate and used to calculate Z′ value for each plate and to normalize the data on a per plate basis. The overall Z score for the campaign were 0.7. Results are reported as percent (%) viability and were calculated using the following formula: % viability=luminescence compound well/median luminescence cell control×100. Four ribavirin positive control wells were included on each plate for quality control purposes, but were not used in Z′ calculations.

For dose response format two dose response curves were calculated for each substance. One assessing cytopathic effect (% CPE Inhibition) at each dose; the other assessing cell viability at each dose. Percent CPE inhibition=100*(1−((luminescence compound well−median luminescence virus control)/(median luminescence cell control-median luminescence virus control. Percent viability=100*luminescence compound well/median luminescence cell control.

The Z factor values were calculated from 1 minus (3* standard deviation of cell control (σc) plus 3* standard deviation of the virus control (σv)/[mean cell control signal (μc) minus mean virus control signal (μv)][7]. The signal/background (S/B) was calculated from mean cell control signal (μc) divided by the mean virus control signal (μv). The signal/noise (S/N) was calculated from mean cell control signal (μc) minus mean virus control signal (μv) divided by the (standard deviation of the cell control signal (σc)2 minus the standard deviation of the virus control signal (σv))½ [7].

An EC₅₀ (for % CPE inhibition) and IC₅₀ (for cell viability) were calculated for each substance using the 4 parameter Levenburg-Marquardt algorithm with parameter A locked at 0 and parameter B locked at 100. Standard deviation, normalized chi2 and hill slope were used to evaluate the curves. Values were not extrapolated beyond the tested range of concentrations.

The criteria for determining compound activity are based on percent inhibition of CPE. As shown above, these compounds are active in the above assay by inhibiting influence-induced CPE. The inhibitions were determined based on percent inhibition of CPE.

### Formulations

Compounds of the present disclosure can be administered by any conventional means available for use in conjunction with pharmaceuticals, either as individual therapeutic agents or in a combination of therapeutic agents. They can be administered alone, but generally administered with a pharmaceutical carrier selected on the basis of the chosen route of administration and standard pharmaceutical practice. The compounds can also be administered in conjunction with other therapeutic agents if desired.

The pharmaceutically acceptable carriers described herein, for example, vehicles, adjuvants, excipients, or diluents, are well-known to those who are skilled in the art. Typically, the pharmaceutically acceptable carrier is chemically inert to the active compounds and has no detrimental side effects or toxicity under the conditions of use. The pharmaceutically acceptable carriers can include polymers and polymer matrices.

The compounds of this disclosure can be administered by any conventional method available for use in conjunction with pharmaceuticals, either as individual therapeutic agents or in a combination of therapeutic agents.

The dosage administered will, of course, vary depending upon known factors, such as the pharmacodynamic characteristics of the particular agent and its mode and route of administration; the age, health and weight of the recipient; the nature and extent of the symptoms; the kind of concurrent treatment; the frequency of treatment; and the effect desired. A daily dosage of active ingredient can be expected to be about 0.001 to 1000 milligrams (mg) per kilogram (kg) of body weight, with the more typical dose being 0.1 to about 30 mg/kg.

Dosage forms (compositions suitable for administration) typically contain from about 1 mg to about 500 mg of active ingredient per unit. In these pharmaceutical compositions, the active ingredient will ordinarily be present in an amount of about 0.5-95% weight based on the total weight of the composition.

The active ingredient can be administered orally in solid dosage forms, such as capsules, tablets, and powders, or in liquid dosage forms, such as elixirs, syrups and suspensions. It can also be administered parenterally, in sterile liquid dosage forms. The active ingredient can also be administered intranasally (nose drops) or by inhalation of a drug powder mist. Other dosage forms are potentially possible such as administration transdermally, via patch mechanism or ointment.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, propylene glycol, glycerin, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

The compounds of the present disclosure, alone or in combination with other suitable components, can be made into aerosol formulations to be administered via inhalation. These aerosol formulations can be placed into pressurized acceptable propellants, such as dichlorodifluoromethane, propane, and nitrogen. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldiallylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl β-aminopropionates, and 2-alkylimidazoline quaternary ammonium salts, and (e) mixtures thereof.

The parenteral formulations typically contain from about 0.5% to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

Pharmaceutically acceptable excipients are also well-known to those who are skilled in the art. The choice of excipient will be determined in part by the particular compound, as well as by the particular method used to administer the composition. Accordingly, there is a wide variety of suitable formulations of the pharmaceutical composition of the present disclosure. The following methods and excipients are merely exemplary and are in no way limiting. The pharmaceutically acceptable excipients preferably do not interfere with the action of the active ingredients and do not cause adverse side-effects. Suitable carriers and excipients include solvents such as water, alcohol, and propylene glycol, solid absorbants and diluents, surface active agents, suspending agent, tableting binders, lubricants, flavors, and coloring agents.

The formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, Eds., 238-250 (1982) and ASHP Handbook on Injectable Drugs, Toissel, 4th ed., 622-630 (1986).

Formulations suitable for topical administration include lozenges comprising the active ingredient in a flavor, usually sucrose and acacia or tragacanth; pastilles comprising the active ingredient in an inert base, such as gelatin and glycerin, or sucrose and acacia; and mouthwashes comprising the active ingredient in a suitable liquid carrier; as well as creams, emulsions, and gels containing, in addition to the active ingredient, such carriers as are known in the art.

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

Suitable pharmaceutical carriers are described in Remington's Pharmaceutical Sciences, Mack Publishing Company, a standard reference text in this field.

The dose administered to an animal, particularly a human, in the context of the present disclosure should be sufficient to affect a therapeutic response in the animal over a reasonable time frame. One skilled in the art will recognize that dosage will depend upon a variety of factors including a condition of the animal, the body weight of the animal, as well as the severity and stage of the condition being treated.

A suitable dose is that which will result in a concentration of the active agent in a patient which is known to affect the desired response. The preferred dosage is the amount which results in maximum inhibition of the condition being treated, without unmanageable side effects.

The size of the dose also will be determined by the route, timing and frequency of administration as well as the existence, nature, and extend of any adverse side effects that might accompany the administration of the compound and the desired physiological effect.

Useful pharmaceutical dosage forms for administration of the compounds according to the present disclosure can be illustrated as follows:

Hard Shell Capsules

A large number of unit capsules are prepared by filling standard two-piece hard gelatine capsules each with 100 mg of powdered active ingredient, 150 mg of lactose, 50 mg of cellulose and 6 mg of magnesium stearate.

Soft Gelatin Capsules

A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

Tablets

A large number of tablets are prepared by conventional procedures so that the dosage unit was 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

Immediate Release Tablets/Capsules

These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

Moreover, the compounds of the present disclosure can be administered in the form of nose drops, or metered dose and a nasal or buccal inhaler. The drug is delivered from a nasal solution as a fine mist or from a powder as an aerosol.

All publications, patents and patent applications cited in this specification are herein incorporated by reference, and for any and all purpose, as if each individual publication, patent or patent application were specifically and individually indicated to be incorporated by reference. In the case of inconsistencies, the present disclosure will prevail.

The foregoing description of the disclosure illustrates and describes the present disclosure. Additionally, the disclosure shows and describes only the preferred embodiments but, as mentioned above, it is to be understood that the disclosure is capable of use in various other combinations, modifications, and environments and is capable of changes or modifications within the scope of the concept as expressed herein, commensurate with the above teachings and/or the skill or knowledge of the relevant art.

The embodiments described hereinabove are further intended to explain best modes known of practicing it and to enable others skilled in the art to utilize the disclosure in such, or other, embodiments and with the various modifications required by the particular applications or uses. Accordingly, the description is not intended to limit it to the form disclosed herein. Also, it is intended that the appended claims be construed to include alternative embodiments.

### REFERENCES

1. Ford, S. M. and J. D. Grabenstein, Pandemics, avian influenza A (H5N1), and a strategy for pharmacists. Pharmacotherapy, 2006. 26(3): p. 312-22.
2. Rogers, D. E., D. B. Louria, and E. D. Kilbourne, The syndrome of fatal influenza virus pneumonia. Trans Assoc Am Physicians, 1958. 71: p. 260-73.
3. Subbarao, K., et al., Characterization of an avian influenza A (H5N1) virus isolated from a child with a fatal respiratory illness. Science, 1998. 279: p. 393-396.
4. Noah, J. W., et al., A cell-based luminescence assay is effective for high-throughput screening of potential influenza antivirals. Antiviral Res, 2007. 73(1): p. 50-9.
5. Noah, D. L., K. Y. Twu, and R. M. Krug, Cellular antiviral responses against influenza A virus are countered at the posttranscriptional level by the viral NS1A protein via its binding to a cellular protein required for the 3′ end processing of cellular pre-mRNAS. Virology, 2003. 307(2): p. 386-95.
6. Eriksson, B., et al., Inhibition of influenza virus ribonucleic acid polymerase by ribavirin triphosphate. Antimicrob Agents Chemother, 1977. 11(6): p. 946-51.
7. Zhang, J., T. Chung, and K. Oldenburg, A simple statistical parameter for use in evaluation and validation of high throughput screening assays. J. Biomol. Screen, 1999. 4: p. 67-73.

1. A compound represented by the one of the following formulas: 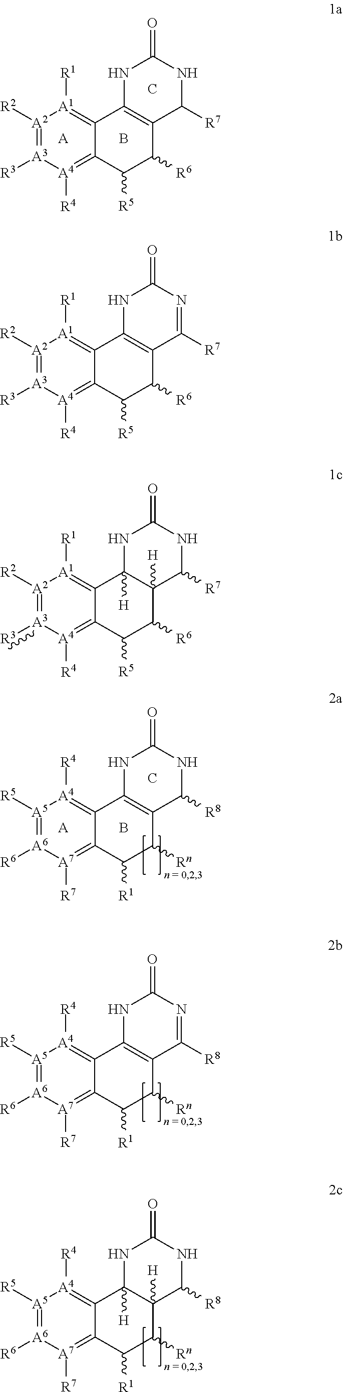 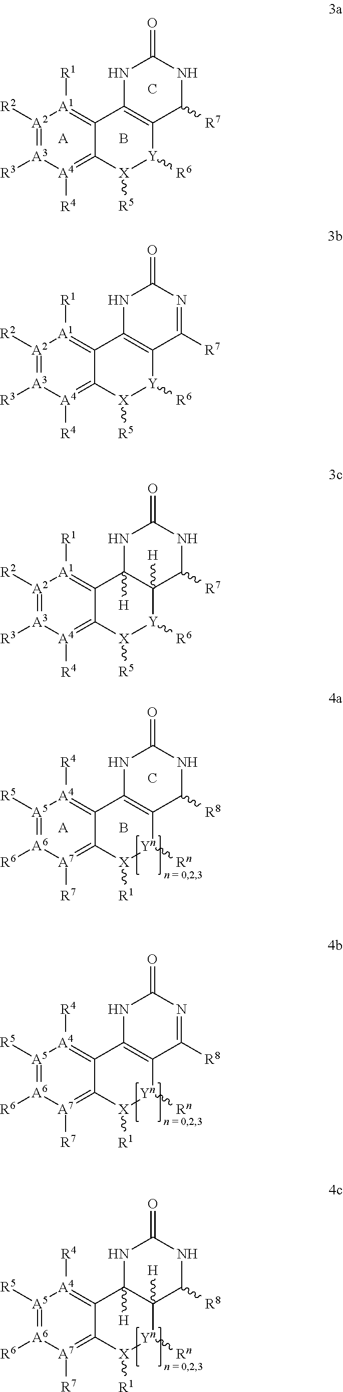 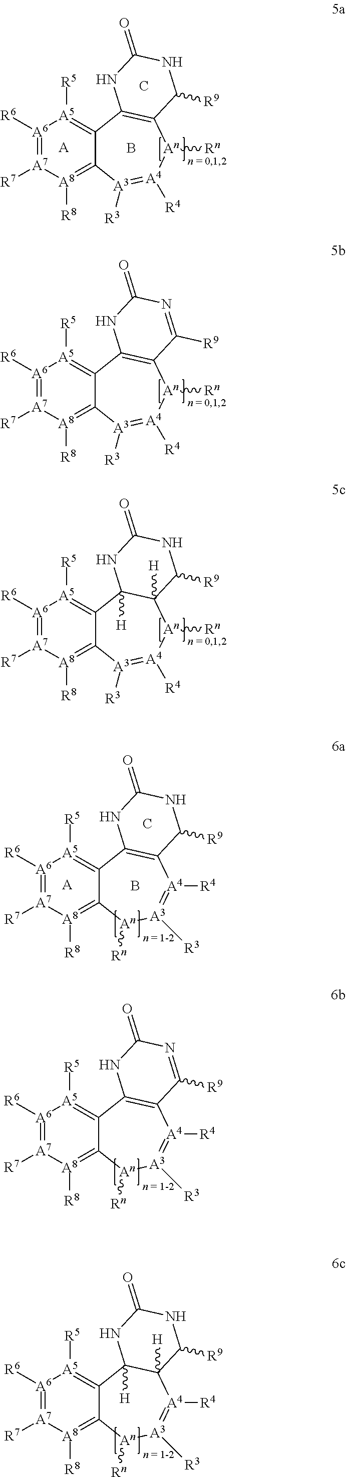
or their stereoisomerically pure forms, or a pharmaceutically acceptable salt thereof, a solvate thereof, a prodrug thereof, and mixtures thereof; wherein,
in formulas 1a, 1b, and 1c:
A¹ thru A⁴ are independently selected from the group consisting C and N such that when any of A¹ thru A⁴ is C, the atom is substituted by the corresponding substituent R¹ thru R⁴;
R¹ thru R⁴ are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino; and
R⁵, R⁶, and R⁷ are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, amino, halogen, azido, cyano, nitro, alkylthio, carboxyl and corresponding esters, carboxamido and amino and mono- or disubstituted amino;
in formulas 2a, 2b, and 2c:
A⁴ thru A⁷ are independently selected from the group consisting of C and N such that when any of A¹ thru A⁴ is C, the atom is substituted by the corresponding substituent R⁴ thru R⁷;
R⁴ thru R⁷ are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino; and
R¹, R², and R³ are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino;
in formulas 3a, 3b, and 3c:
A¹ thru A⁴ are independently selected from the group consisting of C and N such that when any of A¹ thru A⁴ is C, the atom is substituted by the corresponding substituent R¹ thru R⁴;
R¹ thru R⁴ are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino;
X and Y are independently selected from the group consisting of C, N, O, and S, provided that at least one of X or Y is not C;
when X or Y is C or N, the atom is substituted by the corresponding substituent R⁵ thru R⁶;
when bound to C, R⁵ and R⁶ are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino;
when bound to N, R⁵ and R⁶ are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, amino, mono- or disubstituted amino, alkylthio, carbonyl, alkyl- or aryl-substituted carbonyl, carboxyl, alkoxycarbonyl, and aminocarbonyl; and
R⁷ is selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido, amino and mono- or disubstituted amino;
in formulas 4a, 4b, and 4c:
A¹ thru A⁴ are independently selected from the group consisting of C and N such that when any of A¹ thru A⁴ is C, the atom is substituted by the corresponding substituent R¹ thru R⁴;
R¹ thru R⁴ are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino;
X and Y are independently selected from the group consisting of C, N, O, and S, provided that at least one of X or Y is not C;
when X or Y is C or N, the atom is substituted by the corresponding substituent R⁵ thru R⁶;
when bound to C, R⁵ and R⁶ are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino;
when bound to N, R⁵ and R⁶ are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, amino, mono- or disubstituted amino, alkylthio, carbonyl, alkyl- or aryl-substituted carbonyl, carboxyl, alkoxycarbonyl, and aminocarbonyl; and
R⁷ is selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido, amino and mono- or disubstituted amino;
in formulas 5a, 5b, and 5c:
A³ thru A⁸ are independently selected from the group consisting of C and N such that when any of A³ thru A⁸ is C, the atom is substituted by the corresponding substituents R³ thru R⁸;
R³ thru R⁸ are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino;
A¹ and A² are independently selected from the group consisting of C, N, O, and S such that when A¹ or A² is C or N, the atom is substituted by the corresponding substituent R¹ thru R²;
when bound to C, R¹ and R² are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino;
when bound to N, R¹ and R² are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, amino, mono- or disubstituted amino, alkylthio, carbonyl and alkyl- or aryl-substituted carbonyl, carboxyl, alkoxycarbonyl, and aminocarbonyl; and
R⁹ is selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino; and
in formulas 6a, 6b, and 6c:
A³ thru A⁸ are independently selected from the group consisting of C and N such that when any of A³ thru A⁸ are C, the atom is substituted by the corresponding substituent R³ thru R⁸;
R³ thru R⁸ are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino;
A¹ and A² are independently selected from the group consisting of C, N, O, and S such that when A¹ or A² is C or N, the atom is substituted by the corresponding substituent R¹ thru R²;
when bound to C, R¹ and R² are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino;
when bound to N, R¹ and R² are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, amino, mono- or disubstituted amino, alkylthio, carbonyl, alkyl- or aryl-substituted carbonyl, carboxy, alkoxycarbonyl, and aminocarbonyl; and
R⁹ is selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino.

2. A compound according to claim 1 represented by one of the following formulas: 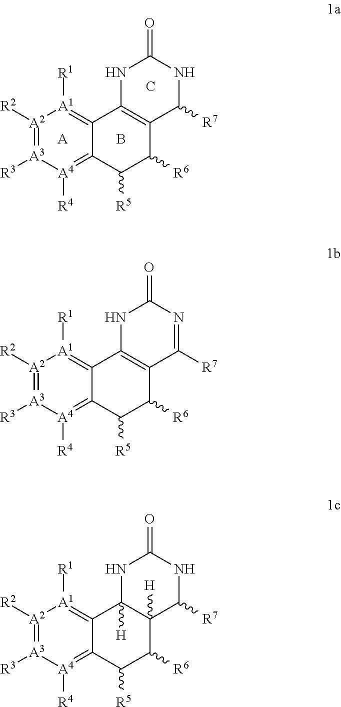
or their stereoisomerically pure forms, or a pharmaceutically acceptable salt thereof, a solvate thereof, a prodrug thereof, and mixtures thereof; wherein
A¹ thru A⁴ are independently selected from the group consisting C and N such that when any of A¹ thru A⁴ is C, the atom is substituted by the corresponding substituent R¹ thru R⁴;
R¹ thru R⁴ are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino; and
R⁵, R⁶, and R⁷ are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, amino, halogen, azido, cyano, nitro, alkylthio, carboxyl and corresponding esters, carboxamido and amino and mono- or disubstituted amino.

3. A compound according to claim 1 represented by one of the following formulas: 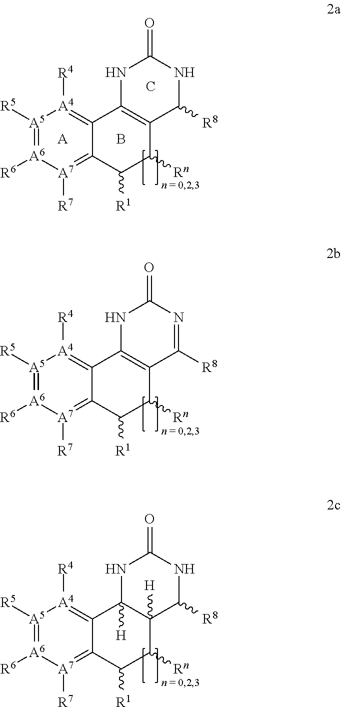
or their stereoisomerically pure forms, or a pharmaceutically acceptable salt thereof, a solvate thereof, a prodrug thereof, and mixtures thereof; wherein
A⁴ thru A⁷ are independently selected from the group consisting of C and N such that when any of A¹ thru A⁴ is C, the atom is substituted by the corresponding substituent R⁴ thru R⁷;
R⁴ thru R⁷ are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino; and
R¹, R², and R³ are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino.

4. A compound according to claim 1 represented by one of the following formulas: 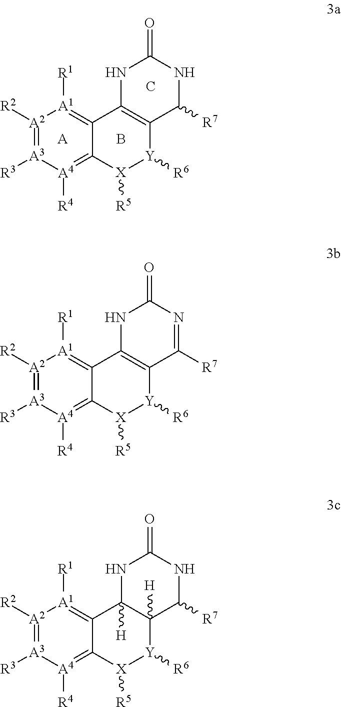
or their stereoisomerically pure forms, or a pharmaceutically acceptable salt thereof, a solvate thereof, a prodrug thereof, and mixtures thereof; wherein
A¹ thru A⁴ are independently selected from the group consisting of C and N such that when any of A¹ thru A⁴ is C, the atom is substituted by the corresponding substituent R¹ thru R⁴;
R¹ thru R⁴ are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino;
X and Y are independently selected from the group consisting of C, N, O, and S, provided that at least one of X or Y is not C;
when X or Y is C or N, the atom is substituted by the corresponding substituent R⁵ thru R⁶;
when bound to C, R⁵ and R⁶ are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino;
when bound to N, R⁵ and R⁶ are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, amino, mono- or disubstituted amino, alkylthio, carbonyl, alkyl- or aryl-substituted carbonyl, carboxyl, alkoxycarbonyl, and aminocarbonyl; and
R⁷ is selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido, amino and mono- or disubstituted amino.

5. A compound according to claim 1 represented by one of the following formulas: 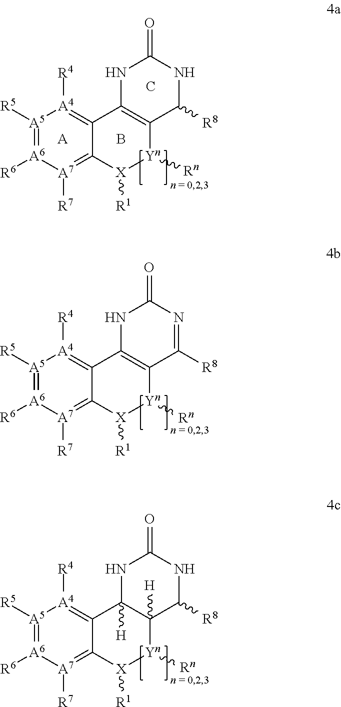
or their stereoisomerically pure forms, or a pharmaceutically acceptable salt thereof, a solvate thereof, a prodrug thereof, and mixtures thereof; wherein
A⁴ thru A⁷ are independently selected from the group consisting of C and N such that when any of A⁴ thru A⁷ is C, the atom is substituted by the corresponding substituent R⁴ thru R⁷;
R⁴ thru R⁷ are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino;
X, Y² and Y³ are independently selected from the group consisting of C, N, O, and S such that when X, Y², or Y³ is C or N, the atom is substituted by the corresponding substituent R¹ thru R³;
when bound to C, R¹, R², and R³ are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino;
when bound to N, R¹, R², and R³ are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, amino, mono- or disubstituted amino, alkylthio, carbonyl, alkyl- or aryl-substituted carbonyl, carboxyl, alkoxycarbonyl, and aminocarbonyl; and
R⁸ is selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino.

6. A compound according to claim 1 represented by the one of the following formulas: 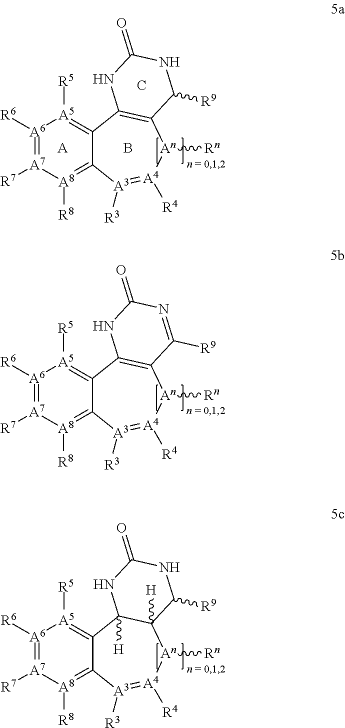
or their stereoisomerically pure forms, or a pharmaceutically acceptable salt thereof, a solvate thereof, a prodrug thereof, and mixtures thereof; wherein
A³ thru A⁸ are independently selected from the group consisting of C and N such that when any of A³ thru A⁸ is C, the atom is substituted by the corresponding substituents R³ thru R⁸;
R³ thru R⁸ are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino;
A¹ and A² are independently selected from the group consisting of C, N, O, and S such that when A¹ or A² is C or N, the atom is substituted by the corresponding substituent R¹ thru R²;
when bound to C, R¹ and R² are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino;
when bound to N, R¹ and R² are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, amino, mono- or disubstituted amino, alkylthio, carbonyl and alkyl- or aryl-substituted carbonyl, carboxyl, alkoxycarbonyl, and aminocarbonyl; and
R⁹ is selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino.

7. A compound according to claim 1 represented by the one of the following formulas: 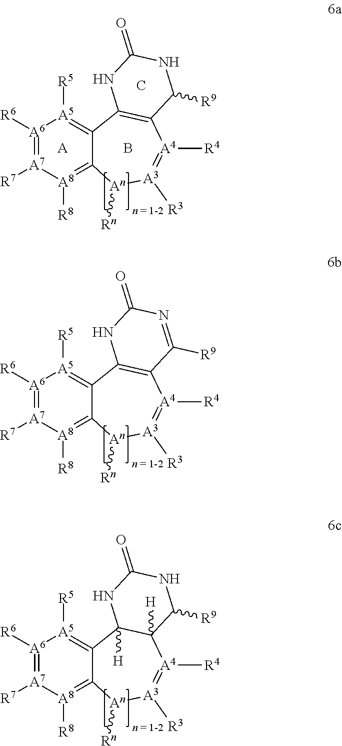
or their stereoisomerically pure forms, or a pharmaceutically acceptable salt thereof, a solvate thereof, a prodrug thereof, and mixtures thereof; wherein
A³ thru A⁸ are independently selected from the group consisting of C and N such that when any of A³ thru A⁸ are C, the atom is substituted by the corresponding substituent R³ thru R⁸;
R³ thru R⁸ are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino;
A¹ and A² are independently selected from the group consisting of C, N, O, and S such that when A¹ or A² is C or N, the atom is substituted by the corresponding substituent R¹ thru R²;
when bound to C, R¹ and R² are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino;
when bound to N, R¹ and R² are independently selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, amino, mono- or disubstituted amino, alkylthio, carbonyl, alkyl- or aryl-substituted carbonyl, carboxy, alkoxycarbonyl, and aminocarbonyl; and
R⁹ is selected from the group consisting of hydrogen, substituted or unsubstituted alkyl, substituted or unsubstituted aryl, substituted or unsubstituted arylalkyl, substituted or unsubstituted cycloalkyl, substituted or unsubstituted heterocyclic, substituted or unsubstituted heteroaryl, hydroxy, alkoxy, carbonyloxy, halogen, azido, cyano, nitro, alkylthio, carboxyl and corresponding esters, carboxamido and amino and mono- or disubstituted amino.

8. A compound selected from those presented in Table I, or a pharmaceutically acceptable salt, solvate, prodrug, or mixture thereof.

9. A pharmaceutical composition comprising a therapeutically effective amount of a compound according to claim 1, and a pharmaceutically acceptable carrier.

10. A method for treating or preventing a viral infection comprising administering to a patient in need thereof a therapeutically effective amount of a compound according to claim 1.

11. The method according to claim 10, wherein the virus is from the Orthomyxoviridae family.

12. The method according to claim 11, wherein the virus is an influenza A virus.

13. The method according to claim 12, wherein the virus is an H1N1 or H5N1 virus.

14. Use of a therapeutically effective amount of a compound according to claim 1 in the manufacture of a medicament for treating or preventing a viral infection.

15. The use according to claim 14, wherein the virus is from the Orthomyxoviridae family.

16. The use according to claim 15, wherein the virus is an influenza A virus.

17. The use according to claim 16, wherein the virus is an H1N1 or H5N1 virus.

18. An method for screening a compound for activity against an influenza virus comprising:
contacting influenza infected-cells with the compound;
measuring the cytopathogenic effect (CPE) of the compound on influenza infected-cells using percent cell viability as the end point; and
calculating the compound's activity.

19. The method according to claim 18, wherein the cell viability is determined from intracellular ATP concentrations.

20. The method according to claim 19, wherein the intracellular ATP concentration is determined using firefly luciferase as a reporter.

21. The method according to claim 18, wherein the percent cell viability is calculated by dividing the luminescence of the influenza virus-infected cells by the luminescence of uninfected control-cells and multiplying by 100.

22. The method according to claim 18, wherein the cells infected with an influenza virus are Madin Darby canine kidney (MDCK) cells.

23. The method according to claim 18, wherein the influenza virus is an H5N1 or H1N1 virus.

24. The method according to claim 18, wherein the calculated activity is reported as an EC₅₀ for % CPE inhibition or IC₅₀ for cell viability.
